(19) United States
(12) Patent Application Publication
(10) Pub. No.: US 2013/0274324 A1
(43) Pub. Date: Oct. 17, 2013

(54) COMPOSITIONS OF FAT-SOLUBLE ACTIVE INGREDIENTS CONTAINING PLANT PROTEIN-SOY POLYSACCHARIDE COMPLEXES

(71) Applicants: Wei Deng, Shanghai (CN); Bruno Leuenberger, Rheinfelden (CH); Olivia Vidoni, Saint Louis (FR); Ping Yao, Shanghai (CN); Baoru Yin, Shanghai (CN)

(72) Inventors: Wei Deng, Shanghai (CN); Bruno Leuenberger, Rheinfelden (CH); Olivia Vidoni, Saint Louis (FR); Ping Yao, Shanghai (CN); Baoru Yin, Shanghai (CN)

(21) Appl. No.: 13/997,471

(22) PCT Filed: Dec. 14, 2011
(86) PCT No.: PCT/EP11/72690

§ 371 Date: Jun. 24, 2013

(30) Foreign Application Priority Data
Dec. 23, 2010 (CN) 201010614218.8

(51) Int. Cl. A23J 3/14; A61K 47/42; A61Q 90/00; A61K 8/64

(52) U.S. Cl. CPC A23J 3/14; A61K 8/645; A61K 47/42; A61Q 90/00
USPC 514/458; 514/773; 514/763; 530/370; 426/656; 426/72; 426/73; 426/540; 426/650; 426/648; 426/590; 426/250

(57) ABSTRACT

The present invention relates to compositions comprising a) 0.1 to 70 weight-% based on the composition of one or more fat-soluble active ingredients; b) one or more plant protein(s) chosen from the group of proteins suitable for food application; and c) one or more soy soluble polysaccharide(s); wherein the sum of the amount of protein(s) and the amount of polysaccharide(s) represents 10 to 85 weight-% based on the composition in dry matter and, wherein the weight ratio of protein(s) to polysaccharide(s) is chosen like 1:b with the proviso that b is comprised between 0.5 and 15.

The present invention relates to compositions comprising one or more plant proteins, one or more soy soluble polysaccharides and one or more fat-soluble active ingredients.

These compositions can be used for the enrichment, fortification and/or coloration of food beverages, animal feed and/or cosmetics. The present invention also refers to the preparation of such compositions. The present invention furthermore refers to a process for the manufacture of a beverage by mixing the compositions with ingredients of beverages. The present invention also refers to beverages obtainable by this process.

Compositions to enrich fortify or color food, beverages, animal feed or cosmetics which contain fat-soluble active ingredients, for example beta-carotene, are known in the art. Beta-Carotene is a preferable colorant compound due to its intense and for the above-mentioned applications very pleasing orange color. Since the final products in which these colorants, nutrients, and/or additives are used are usually aqueous compositions such as beverages, additional compounds have to be added to avoid separation of fat (oil) phases in the product, which would render the corresponding product unacceptable.

Therefore, fat-soluble active ingredients are often combined with auxiliary compounds, such as starches or fish gelatin, in order to prevent phase separation in the final aqueous composition. Those auxiliary compounds, however, often have a negative influence on the color properties and the nutritional properties of the final products. It is therefore desired to develop new compositions of fat-soluble active ingredients, which contain improved auxiliary compounds, which have very good properties referring to taste, emulsification, emulsion stability, film forming ability and/or color of the final product in which it is used.

Proteins have been used as emulsifiers in food products for many years [E. Dickinson, D. J. McClements, Molecular basis of protein functionality, in: E. Dickinson, D. J. McClements (Eds.), Advances in Food Colloids, Blackie Academic & Professional, London, UK, 1995, pages 26-79]. However, the emulsification capacity may be lost at or near the isoelectric point, i.e. at a certain protein specific pH at which the net charges and solubility of the particular protein are minimal. Furthermore the emulsion stability decreases due to the screening of the electrostatic repulsion of protein in the presence of high concentration of salts. Most proteins have an isoelectric point below pH 7. Most foods and beverages are acidic; therefore the poor emulsion stability at the isoelectric point limits the applicability of proteins in food and beverage industries.

The stability of protein containing oil-in-water emulsions depends strongly on the charge density and structure of the emulsifier adsorbed on the emulsion droplet surface. The protein adsorption layers prevent the drop-drop coalescence by stabilizing the emulsion films. However, protein-stabilized emulsions are highly sensitive to environmental stresses such as pH and ionic strength [Rungnaphar Pongsawatmanit, Thepkunya Harnsilawat, David J. McClements, Colloids and Surfaces A: Physicochem. Eng. Aspects, 287, 59-67, 2006]. When the aqueous pH approaches the isoelectric point of a protein and/or the salt concentration is high, the electrostatic repulsion of the protein layers decreases and, therefore, protein precipitation, emulsion droplet coalescence and creaming occur [Eric Dickinson Soft Matter, 2008, 4, 932-942].

Proteins as emulsifiers do not function effectively at pH values close to their isoelectric point because they precipitate [N. G. Diftisa, C. G. Biliaderisb, V. D. Kiosseoglou, Food Hydrocolloids 19 (2005) 1025-1031].

The emulsion stability may be improved by forming protein-polysaccharide conjugates produced through covalent binding [Eric Dickinson Soft Matter, 2008, 4, 932-942]. The protein-polysaccharide conjugates have improved emulsifying and steric stabilizing properties, especially under conditions where the protein alone has poor solubility [Eric Dickinson Soft Matter, 2008, 4, 932-942].

The improvement of emulsifying properties of soybean protein by conjugation with polysaccharide has also been reported [N. Diftis and V. Kiosseoglou, FoodChemistry, 81, 1, 2003; N. Diftis and V. Kiosseoglou, Food Hydrocolloids, 20, 787, 2006; N. G. Diftis, et al., Food Hydrocolloids, 19, 1025, 2005; N. Diftis and V. Kiosseoglou, Food Chemistry, 96, 228]. Protein-polysaccharide conjugations can improve emulsifying properties of proteins, especially through oil droplet size reduction and emulsion stabilization. These conjugates can be produced by Maillard-type reactions between protein and polysaccharide, or by other reactions. Xu and Yao, (Langmuir 2009, 25 (17), 9714-9720) have also described oil-in-water emulsions prepared from soy protein-dextran conjugates. The conjugates are adsorbed at the interface together with unreacted protein constituents, enhancing steric stabilization forces of oil droplets. However, the Maillard type reaction is a time-consuming process that is poorly amenable to industrial scale reaction. Therefore, the use of such conjugates remains inadequate in food and beverage applications.

Therefore, there is still a need for compositions comprising fat-soluble active ingredients for the enrichment, fortification and/or coloration of food, beverages, animal feed, cosmetics or pharmaceutical compositions which do not show the above-mentioned problems.

It was therefore an object of the present invention to provide compositions of fat-soluble active ingredients having the desired properties as indicated above, e.g. very good properties referring to optical clarity and emulsion stability and/or an improved color intensity and color stability (wherever applicable). It was also an objective of the invention to improve the process for the preparation of compositions of fat-soluble active ingredients.

This objective has been solved by a composition comprising:
a) 0.1 to 70 weight-% based on the composition of one or more fat-soluble active ingredients, preferably 0.1 to 30 weight-%;
b) one or more plant protein(s) chosen from the group of proteins suitable for food application; and
c) one or more soy soluble polysaccharide(s);
   wherein the sum of the amount of protein(s) and the amount of polysaccharide(s) represents 10 to 85 weight-% based on the composition in dry matter, preferably 25 to 85 weight-%, more preferably, 35 to 85 weight-% and wherein the weight ratio of protein(s) to polysaccharide(s) is chosen like 1:b with the proviso that b is comprised between 0.5 and 15.

As used herein, the term “fat-soluble active ingredient” refers to vitamins selected from the group consisting of vitamin A, D, E, K and derivatives thereof; polyunsaturated fatty acids; lipophilic health ingredients; carotenoids; and flavoring or aroma substances as well as mixtures thereof.

Polyunsaturated fatty acids (PUFAs), which are suitable according to the present invention, are mono- or polyunsaturated carboxylic acids having preferably 16 to 24 carbon atoms and, in particular, 1 to 6 double bonds, preferably having 4 or 5 or 6 double bonds.

The unsaturated fatty acids can belong both to the n-6 series and to the n-3 series. Preferred examples of n-3 polyunsaturated acids are eicosapenta-5,8,11,14,17-enoic acid and docosahexa-4,7,10,13,16,19-enoic acid; preferred examples of a n-6 polyunsaturated acid are arachidonic acid and gamma linolenic acid.

Preferred derivatives of the polyunsaturated fatty acids are their esters, for example glycerides and, in particular, triglycerides; particularly preferably the ethyl esters. Triglycerides of n-3 and n-6 polyunsaturated fatty acids are especially preferred.

The triglycerides can contain 3 uniform unsaturated fatty acids or 2 or 3 different unsaturated fatty acids. They may also partly contain saturated fatty acids.

When the derivatives are triglycerides, normally three different n-3 polyunsaturated fatty acids are esterified with glycerin. In one preferred embodiment of the present invention triglycerides are used, whereby 30% of the fatty acid part is n-3 fatty acids and of these, 25% are long-chain polyunsaturated fatty acids. In a further preferred embodiment commercially available ROPUFA® ‘30’ n-3 Food Oil (DSM Nutritional Products Ltd, Kaiseraugst, Switzerland) is used.

In another preferred embodiment of the present invention, the PUFA ester is ROPUFA® ‘75’ n-3 EE. ROPUFA ‘75’ n-3 EE is refined marine oil in form of an ethyl ester with minimum content of 72% n-3 fatty acid ethyl ester. It is stabilized with mixed tocopherols, ascorbyl palmitate, citric acid and contains rosemary extract.

In another preferred embodiment of the present invention the PUFA ester is ROPUFA® ‘10’ n-6 Oil, a refined evening primrose oil with minimum 9% gamma linolenic acid which is stabilized DL-alpha-tocopherol and ascorbyl palmitate.

According to the present invention it can be advantageous to use naturally occurring oils (one or more components) containing triglycerides of polyunsaturated fatty acids, for example marine oils (fish oils) and/or plant oils, but also oils extracted from fermented biomass or genetically modified plants

Preferred oils which comprise triglycerides of polyunsaturated fatty acids are olive oil, sunflower seed oil, evening primrose seed oil, borage oil, grape seed oil, soybean oil, groundnut oil, wheat germ oil, pumpkin seed oil, walnut oil, sesame seed oil, rapeseed oil (canola), blackcurrant seed oil, kiwifruit seed oil, oil from specific fungi and fish oils.

Preferred examples for polyunsaturated fatty acids are e.g. linoleic acid, linolenic acid, arachidonic acid, docosahexaenic acid, eicosapentaenic acid and the like.

According to the present invention preferred lipophilic health ingredients are resveratrol; ligusticum; ubichinones and/or ubiquinols (one or more components) selected from coenzyme Q 10 (also referred to as “CoQ10”), coenzyme Q 9, and/or their reduced forms (the corresponding ubiquinols); genistein and/or alpha-lipoic acid.

Especially preferred fat-soluble active ingredients of the invention are carotenoids, especially beta-carotene, lycopene, lutein, bixin, astaxanthin, apocarotenal, beta-apo-8′-carotenal, beta-apo-12′-carotenal, canthaxanthin, cryptoxanthin, citranaxanthin and zeaxanthin. Most preferred is beta-carotene.

In an preferred embodiment of the invention, the composition comprises between 0.1 and 70 weight-%, further preferred between 0.1 and 30 weight-%, further preferred between 0.2 and 20 weight-%, most preferred between 0.5 and 15 weight-% of one or more fat-soluble active ingredients, based on the total composition.

According to the present invention preferred plant protein (s) are derived from soy, lupin (e.g. L. albus, L. angustifolius or varieties thereof), pea and/or potato. The proteins may be isolated from any part of the plant, including fruits (like e.g. soy beans), seeds (including prepared or processed seeds) and the like; or from whole flour or defatted products such as shred, flakes etc.

For the composition of the present invention, especially preferred are soy and pea protein, even more preferred soy protein is “acid soluble soy protein” (Soyasour 4000K, with a protein content greater or equal to 60 weight-%). Most preferred is Soyasour 4000K, with a protein content greater or equal to 80 weight-%, moisture, below or equal to 7.5 weight-%, fat below or equal to 1.5 weight-%, pH 3.6 to 6.4) It can be sourced from Jilin Fuji Protein Co. Ltd. Preferred Pea protein source is from Cosucra SA (Warcoing, Belgium)

The term “soy soluble polysaccharide” as used herein refers to Soy soluble polysaccharide with a content greater or equal to 60 weight-% polysaccharides. Most preferred soy soluble polysaccharide is soy soluble polysaccharide with a content greater or equal to 70 weight-% polysaccharides, smaller or equal to 10 weight-% protein, smaller or equal to 1 weight-% fat, smaller or equal to 8 weight-% moisture, smaller or equal to 8 weight-% ash, and a pH comprised between 3 to 6. It can be sourced from Fuji Co., Ltd.

It is preferred to choose the weight ratio of protein(s) to polysaccharide(s) like 1:b with the proviso that b is comprised between 0.5 and 15, especially preferred b is chosen from the range of from 1 to 7, more preferred from 3 to 7, most preferred from 4 to 5.

In an especially preferred embodiment of the present invention stable protein-soy soluble polysaccharide emulsifiers are formed by subsequent heating of the emulsion.

Accordingly, the invention also relates to a process for the manufacture of a stable emulsifier composition as indicated above comprising the following steps (the process can be carried out using the ingredients in amounts as specified herein):
I) suspending the protein in water;
II) optionally removing not-dissolved protein from the suspension of step I);
III) mixing one or more soy soluble polysaccharide(s) in a weight ratio of protein(s) to polysaccharide(s) of from 1:0.5 to 1:15;
IV) adjusting the pH to a value comprised between 3 and 5 such that protein-polysaccharides electrostatic complexes are formed
V) adding the organic phase, comprising the one or more fat-soluble active ingredients to the complex;
VI) homogenizing the mixture of step V) with a conventional emulsification process known to the person skilled in the art.
VII) heating the emulsion at a temperature comprised between 70 to 95° C., preferably 80 to 90° C. for at least 45 minutes, preferably, at least 1 hour
VIII) optionally drying the emulsion of step VII).

According to the present invention preferred proteins are plant proteins as described above.

The drying step may be carried out with any conventional drying process known to the person skilled in the art, preferred are spray drying and/or a powder catch process where sprayed suspension droplets are caught in a bed of an adsorbant such as starch or calcium silicate or silicic acid or calcium carbonate or mixtures thereof and subsequently dried.

The emulsion of step VII) may be used as it is or dried for later use.

Homogenization can be performed with standard emulsification techniques like ultrasonication or high pressure homogenization (800 to 1200 bar).

Ultrasonication generates alternating low-pressure and high-pressure waves in liquids, leading to the formation and violent collapse of small vacuum bubbles. This phenomenon, called cavitation, causes high speed impinging liquid jets and strong hydrodynamic shear-forces, combined with compression, acceleration, pressure drop, and impact, causing the disintegration of particles and dispersion throughout the product as well as the mixing of reactants. (Encyclopedia of emulsion technology, 1983, Vol 1, P. Walstra, page 57, Ed P. Becher, ISBN: 0-8247-1876-3)

In the case of the high pressure homogenization process, the mixture containing already the organic and the aqueous phases is passed through a gap in the homogenizing valve; this creates conditions of high turbulence and shear, combined with compression, acceleration, pressure drop, and impact, causing the disintegration of particles and dispersion throughout the product. The size of the particles depends on the operating pressure used during the process and the type of gap selected. (Food and Bio Process Engineering, Dairy Technology, 2002, H. G. Kessler, Ed A. Kessler, ISBN 3-9802378-5-0).

The most preferred homogenization to carry out the present invention is high pressure homogenization according to (Donsi et al. J. Agric. Food Chem., 2010, 58:10653-10660) in view of the efficiency and high throughput of this technology to produce nanoemulsions.

The present invention also relates to a plant protein-soy soluble polysaccharide complex obtainable by a process as described above, and preferably, wherein the plant protein is soy protein or pea protein.

The present invention is also directed to the use of compositions as described above for the enrichment, fortification and/or coloration of food, beverages, animal feed and/or cosmetics, preferably for the enrichment, fortification and/or coloration of beverages.

Other aspects of the invention are food, beverages, animal feed, cosmetics containing a composition as described above.

Beverages wherein the product forms of the present invention can be used as a colorant or an additive ingredient can be carbonated beverages e.g., flavored seltzer waters, soft drinks or mineral drinks, as well as non-carbonated beverages e.g. flavored waters, fruit juices, fruit punches and concentrated forms of these beverages. They may be based on natural fruit or vegetable juices or on artificial flavors. Also included are alcoholic beverages and instant beverage powders. Besides, sugar containing beverages diet beverages with non-caloric and artificial sweeteners are also included.

Further, dairy products, obtained from natural sources or synthetic, are within the scope of the food products wherein the product forms of the present invention can be used as a colorant or as a nutritional ingredient. Typical examples of such products are milk drinks, ice cream, cheese, yogurt and the like. Milk replacing products such as soymilk drinks and tofu products are also comprised within this range of application.

Also included are sweets which contain the product forms of the present invention as a colorant or as an additive ingredient, such as confectionery products, candies, gums, desserts, e.g. ice cream, jellies, puddings, instant pudding powders and the like.

Also included are cereals, snacks, cookies, pasta, soups and sauces, mayonnaise, salad dressings and the like which contain the product forms of the present invention as a colorant or a nutritional ingredient. Furthermore, fruit preparations used for dairy and cereals are also included.

The final concentration of the one or more fat-soluble active ingredients, preferred carotenoids, especially beta-carotene, which is added via the compositions of the present invention to the food products may preferably be from 0.1 to 50 ppm, particularly from 1 to 30 ppm, more preferred 3 to 20 ppm, e.g. about 6 ppm, based on the total weight of the food composition and depending on the particular food product to be colored or fortified and the intended grade of coloration or fortification.

The food compositions of this invention are preferably obtained by adding to a food product the fat-soluble active ingredient in the form of a composition of this invention. For coloration or fortification of a food or a pharmaceutical product a composition of this invention can be used according to methods per se known for the application of water dispersible solid product forms.

In general the composition may be added either as an aqueous stock solution, a dry powder mix or a pre-blend with other suitable food ingredients according to the specific application. Mixing can be done e.g. using a dry powder blender, a low shear mixer, a high-pressure homogenizer or a high shear mixer depending on the formulation of the final application. As will be readily apparent such technicalities are within the skill of the expert.

The invention also relates to a process for the manufacture of a beverage comprising the steps of homogenizing the composition according to the present invention, and mixing 1 to 50 ppm based on the fat soluble content, preferably 5 ppm of the emulsion with further usual ingredients of beverages.

Further, the present invention relates to beverages obtainable by the process for the manufacture of a beverage as described above.

### BRIEF DESCRIPTION OF THE FIGURES

FIG. 1 shows real time DLS (dynamic light scattering) result of emulsions digested for 170 minutes.

FIG. 2 shows (A) ζ-potential of pea protein solution and supernatant as a function of pH. (B) ζ-potential of individual pea protein and soy polysaccharide solutions as a function of pH.

FIG. 3 shows the visible spectra of β-carotene emulsion before and after the addition of FeCl₃.

FIG. 4 shows the visible spectra of β-carotene emulsion before and after the addition of FeCl₃. The emulsion was digested by typsin and pectinase.

The present invention is further illustrated by the following examples, which are not intended to be limiting.

### EXAMPLES

### Example 1

### Production of A Stable Emulsion With Soy Protein/Soy Soluble Polysaccharide

### Materials

Soy protein is from Jilin Fuji Protein Co. Ltd. (Soyasour 4000K, acid soluble soy protein; ASSP) with protein content 88% (dry basis). It has an isoelectric point around pH 4.7. Soy soluble polysaccharides (SSP) with 70 to 80 weight-% polysaccharides were sourced from Fuji Co., Ltd.

### Preparation of Protein/Soy Soluble Polysaccharide Emulsion

The preparation of the complexes is performed in situ in two steps: first mixing both products (ASSP and SSP) before homogenization and then heating the emulsion to 80° C. to fix the structure created. The acid soluble soy protein (ASSP) and soy soluble polysaccharide (SSP) are mixed at pH 3.25, and stirred 3 to 4 hours. Soybean oil was then added into the aqueous mixture solution of ASSP-SSP. The resulting solution was homogenized at room temperature with a homogenizer (FJ200-S, Shanghai Specimen Model Co) at 10000 rpm for 1 min. and immediately homogenized at 800 bar for 2.5 min. (AH100D, ATS engineering Inc). Finally, the emulsion was heated at 80° C. for 1 hour.

### Particle Size Measurement

Freshly diluted emulsion samples with the same pH and NaCl concentration were used for every dynamic light scattering (DLS) measurement. The measurements were carried out on a Malvern Autosizer 4700 (Malvern Instruments, Worcs, UK) equipped with a multi-τ digital time correlator (Malvern PCS7132) and a solid-state laser (Compass 315M-100, Coherent Inc.; output power≈100 mW, λ=532 nm). The measurements were performed at 25° C. and a fixed scattering angle of 90°. The measured time correlation functions were analyzed by Automatic Program equipped with the correlator. The particle size (z-average hydrodynamic diameter, Dₕ) was obtained by automatic mode analysis. Two batches of samples were measured and averaged data was reported.

### Example 2

### Stability of the Emulsion At High Salt Concentration

The soy protein ASSP and soy soluble polysaccharide SSP were mixed at pH 3.25. The homogenization condition is as follows: protein concentration 5 mg/ml, weight ratio of protein to polysaccharides 1:5, 10% oil volume fraction, 800 bar homogenization for 2.5 minutes, follow by a heating process or not. After overnight storage at 4° C., the pH of the resulting emulsions was adjusted to 5.0 or 6.0, and NaCl was added, then, the emulsions were kept at 4° C. to investigate long-term stability. The droplet size distribution of the emulsions was measured by dynamic light scattering (DLS). The DLS samples were prepared by diluting the emulsions with freshly prepared aqueous solution having the same pH value and the same salt concentration.

After the heating process, the droplet size distributions of the emulsions do not change significantly compared with the emulsions without heating. However, their stabilities are different as shown in Table 1 and Table 2. The heated emulsions exhibit a long-term stability in pH 5.0 and 6.0 media containing salt.

TABLE 1
Particle size of freshly prepared emulsions/heated emulsions at pH 5
and pH 6 at different sodium chloride concentrations. The experiment
was repeated with two different batches labelled (1) and (2) to assess
reproducibility of the data.

| | | Particle Size, nm | | | | |
|---|---|---|---|---|---|---|
| NaCl concentration (M) | | 0.02 | 0.05 | 0.1 | 0.15 | 0.20 |
| Emulsion at pH 5 | (1) | 285 | 301 | 365 | 396 | 406 |
| | (2) | 284 | 309 | 378 | 403 | 412 |
| Heated emulsion at pH 5 | (1) | 255 | 268 | 292 | 293 | 306 |
| | (2) | 263 | 267 | 294 | 299 | 307 |
| Emulsion at pH 6 | (1) | 343 | 363 | 435 | 460 | 486 |
| | (2) | 346 | 372 | 434 | 466 | 489 |
| Heated emulsion at pH 6 | (1) | 286 | 276 | 294 | 297 | 301 |
| | (2) | 287 | 279 | 298 | 304 | 299 |

TABLE 2
Particle size of emulsions/heated emulsions at pH 5 and pH 6
at different sodium chloride concentrations after 108 days of
storage. The experiment was repeated with two different
batches labelled (1) and (2) to assess reproducibility of the
data. The unheated emulsions in pH 5 and 6 media containing
0.15 and 0.2M NaCl presented creaming during the storage.
The heated emulsions were homogenous in appearance
after the storage.

| | | Particle Size, nm | | | | |
|---|---|---|---|---|---|---|
| NaCl concentration (M) | | 0.02 | 0.05 | 0.1 | 0.15 | 0.20 |
| Emulsion at pH 5 | (1) | 295 | 329 | 353 | 350 | 628 |
| | (2) | 301 | 333 | 359 | 387 | 625 |
| Heated emulsion at pH 5 | (1) | 289 | 291 | 309 | 311 | 322 |
| | (2) | 284 | 287 | 308 | 311 | 334 |
| Emulsion at pH 6 | (1) | 383 | 372 | 513 | 702 | 805 |
| | (2) | 397 | 406 | 547 | 709 | 827 |
| Heated emulsion at pH 6 | (1) | 287 | 301 | 321 | 353 | 344 |
| | (2) | 285 | 304 | 328 | 351 | 350 |

### Example 3

### Stability of the Emulsion At Different pH

The emulsions were prepared at pH 3.25, with a protein concentration of 5 mg/ml, and a weight ratio of protein to polysaccharides 1:5, 10% oil volume fraction, 800 bar homogenization for 2.5 minutes, heating at 80° C. for 1 h or without heating. Then, the pH of the emulsions was adjusted to different values and the emulsions were stored at 4° C. to investigate the stability. For the emulsions that underwent heat process, the emulsions are homogenous in the pH range of 2-8 after 140 and 145 days of storage. However, creaming appeared for the unheated emulsions in pH 7 and 8 medium after 20 days of storage; later, creaming also happened for the unheated emulsions in pH 2 and pH 6 medium. Table 3 shows that the sizes increase at pH 2 and also increase from pH 5 to 8; the unheated emulsions increase much more than the heated emulsions before and after the storage. The result shown in Table 3 further confirms that the heating process is necessary to increase the stability of the emulsions prepared by high pressure homogenization.

TABLE 3
Particle size of emulsions at different pH of freshly prepared and of emulsions
stored for 140 to 145 days. The experiment was repeated with two different batches
labelled (1) and (2) to assess reproducibility of the data.

| | | Particle Size, nm | | | | | | | |
|---|---|---|---|---|---|---|---|---|---|
| | | pH | | | | | | | |
| | | 3.25 | 2 | 3 | 4 | 5 | 6 | 7 | 8 |
| Fresh Emulsion | (1) | 238 | 400 | 239 | 239 | 263 | 327 | 457 | 507 |
| | (2) | 227 | 434 | 230 | 224 | 283 | 338 | 428 | 481 |
| Emulsion after | (1) | 254 | 634 | 262 | 271 | 289 | 425 | 571 | 654 |
| 145 days storage | | | | | | | | | |
| Emulsion after | (2) | 256 | 753 | 248 | 230 | 272 | 412 | 583 | 643 |
| 140 days storage | | | | | | | | | |
| Fresh Heated | (1) | 245 | 263 | 245 | 239 | 256 | 280 | 314 | 321 |
| emulsion | (2) | 232 | 283 | 236 | 229 | 262 | 284 | 299 | 314 |
| Heated emulsion | (1) | 264 | 293 | 266 | 276 | 289 | 312 | 344 | 378 |
| after 145 days | | | | | | | | | |
| storage | | | | | | | | | |
| Heated emulsion | (2) | 257 | 304 | 243 | 250 | 275 | 312 | 340 | 367 |
| after 140 days | | | | | | | | | |
| storage | | | | | | | | | |

### Example 4

### Particle Size of Freshly Prepared Heated Emulsions Performed With Soy Protein Alone Or Soy Soluble Polysaccharides Alone At Different pH

The stability of the emulsions prepared with individual acid soluble soy proteins (ASSP) and individual soy soluble polysaccharides (SSP) were also investigated (see Table 4) in comparison with ASSP/SSP complex emulsions. The emulsions were prepared at the condition of pH 3.25, protein concentration 5 mg/ml or polysaccharide concentration 25 mg/ml, 10% oil volume fraction, 800 bar homogenization for 2.5 minutes, heating at 80° C. for 1 h. Then, the pH of the emulsions was adjusted to different values and the emulsions were stored at 4° C. For fresh ASSP emulsions, creaming appeared at pH range of 5-8. After 1 week of storage, creaming appeared for all of the samples, except ASSP emulsion at pH 3.25. This result supports the conclusion that the ASSP/SSP complex emulsions are superior to individual ASSP and SSP emulsions.

TABLE 4
Particle size of freshly prepared SSP or ASSP emulsions at different
pH. The experiment was repeated with two different batches labelled
(1) and (2) to assess reproducibility of the data.

| | | Particle Size, nm | | | | | | |
|---|---|---|---|---|---|---|---|---|
| | | pH | | | | | | |
| | | 3.25 | 3 | 4 | 5 | 6 | 7 | 8 |
| Heated SSP | (1) | 519 | 630 | 1024 | 1088 | 997 | 980 | 834 |
| emulsion | (2) | 544 | 609 | 863 | 975 | 908 | 854 | 817 |

| | | | | | |
|---|---|---|---|---|---|
| Heated ASSP | (1) | 247 | 268 | 313 | creaming |
| emulsion | (2) | 254 | 278 | 316 | creaming |

### Example 5

### Influence of Protein/Polysaccharide Complexation pH On Homogenization

Homogenization was performed at different pH values to investigate the influence of the complex formation on the stability of the emulsions. The homogenization condition is as follows: adjusting ASSP and SSP solutions to desired pH, mixing ASSP and SSP solutions with the same pH, protein concentration 5 mg/ml, weight ratio of protein to polysaccharides 1:5, 10% oil volume fraction, 800 bar homogenization for 3-4 minutes. The results shown in Table 5 and 6 indicate that homogenization in the pH range of 3-4 can produce stable emulsions. In this pH range, ASSP and SSP form electrostatic complexes, indicating that the complex formation is essential to produce the droplets with the structure of ASSP/SSP complex membrane in oil-water interface, and a SSP shell that stabilizes the droplets in aqueous solution.

TABLE 5
DLS result of fresh emulsions homogenized at different pH. The experiment was
repeated with two different batches labelled (1) and (2) to assess reproducibility of the
data.

| | | Homogenizing pH | | | | | | | | |
|---|---|---|---|---|---|---|---|---|---|---|
| | | 3 | 3.25 | 3.50 | 3.75 | 4 | 5 | 6 | 7 | 8 |
| Particle Size, (nm) | (1) | 224 | 232 | 227 | 236 | 242 | 393 | — | — | — |
| | (2) | 223 | 224 | 230 | 231 | 232 | 493 | 4532 | 4621 | 2132 |

TABLE 6
DLS result of the emulsions shown in Table 5 after 2 months of
storage. The experiment was repeated with two different batches
labelled (1) and (2) to assess reproducibility of the data.

| | | Homogenizing pH | | | | | | | | |
|---|---|---|---|---|---|---|---|---|---|---|
| | | 3 | 3.25 | 3.50 | 3.75 | 4 | 5 | 6 | 7 | 8 |
| Parti- | (1) | 220 | 229 | 223 | 235 | 211 | Creaming | — | — | — |

| | | | | | | | |
|---|---|---|---|---|---|---|---|
| cle | (2) | 225 | 228 | 227 | 219 | 220 | Creaming |
| Size, | | | | | | | |
| (nm) | | | | | | | |

### Example 6

### Digestion of the SSP In the Emulsion With Enzyme

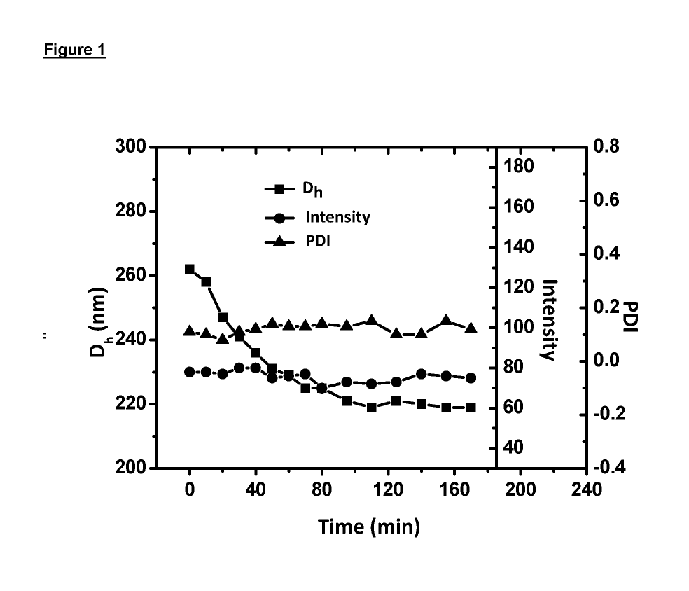

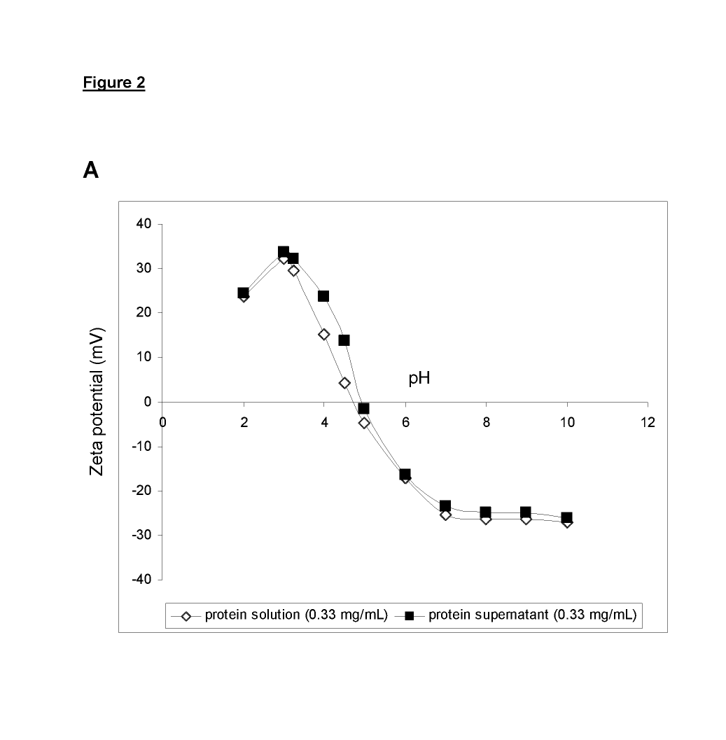

The emulsion was prepared at pH 3.25, protein concentration 5 mg/ml, weight ratio of protein to polysaccharides 1:5, 10% oil volume fraction, 800 bar homogenization for 2.5 minutes, and heating at 80° C. for 1 h as in example 1. Then, the soy polysaccharide in the emulsion was hydrolyzed by pectinase. For monitoring the size change by real time DLS measurement, the hydrolysis was performed at 25° C. and pH 5.0 to reduce the hydrolysis rate. During the DLS measurement, a 3 μL of 0.5% pectinase solution was added into a polystyrene cuvette containing diluted emulsion which was prepared by diluting 5 μL of original emulsion with 3 mL of pH 5.0 aqueous solution. Then, the droplet size was measured every 10 min for the first 80 min, and every 15 min for another 90 min. The data as shown (FIG. 1) reveal that droplet size decreases from 262 nm to 219 nm and levels off. From the decrease of the Dₕ value before and after the digestion, we can estimate that the polysaccharide layer of the droplets is about 22 nm.

### Example 7

### Solubility of Pea Protein In Aqueous Solution

Pea protein ((Pisane® F9) from CosucroSA Belgium) was dissolved in water with an apparent concentration of 22 mg/mL. The solution was adjusted to pH 1 to 10 with NaOH or HCl solution. After equilibrium overnight, the solutions were centrifuged at 5000 rpm or 7800 rpm for 30 min. The supernatants were lyophilized and then the powders were weighed to estimate the solubility of pea protein in different pH. The data is shown in Table 7.

TABLE 7
Solubility of pea protein in the pH range of 1 to 10. The original
concentration of pea protein was 22 mg/mL.

Protein concentration of supernatant (mg/mL)

| pH | Centrifugation at 7800 rpm | Centrifugation at 5000 rpm |
|---|---|---|
| 1 | 13.2 | 19.2 |
| 2 | 13.8 | 19.8 |
| 3 | 5.0 | 16.0 |
| 4 | 2.4 | 2.4 |
| 5 | 2.4 | 2.2 |
| 6 | 3.4 | 3.8 |
| 6.8 | — | 16.0 ± 0.9^{a} |
| 7 | 12.8 | 17.8 |
| 8 | 12.4 | 18.8 |
| 9 | 12.8 | 18.5 |
| 10 | 13.9 | 19.4 |

^{a}The original pH of the protein aqueous solution is 6.8; the datum represents a mean value (n = 6).

Table 7 shows that the protein concentrations are about 2 mg/mL at pH 4 and 5, where is close to the isoelectric point of pea protein. At pH 1-3 and 7-10, after centrifugation at 5000 rpm, the protein concentrations are about 40% higher than those after centrifugation at 7800 rpm. This result indicates that the major part of the protein is dispersible aggregates. The solubility of pea protein at pH 3.0 and 4.0 is much smaller than acid soluble soy protein, which remains 20 mg/mL after 10000 rpm centrifugation at pH 3.0 and 4.0 for 23 mg/mL of original solution.

We further prepared pea protein solution of 50 mg/mL, then, changed the pH to 3.0 and 3.25 to investigate the solubility. The data in Table 8 reveal that at pH 3.25, after centrifugation at 5000 rpm for 30 min, about 67% of the protein aggregates were removed.

TABLE 8
Solubility of pea protein at pH 3.0 and 3.25 solution. The original protein
concentration was 50 mg/mL. The protein solution was adjusted to
pH 3.0 or 3.25 then centrifugation for 30 min.

Protein concentration (mg/mL)

| pH | Centrifugation at 7800 rpm | Centrifugation at 5000 rpm |
|---|---|---|
| 3.0 | 18.2 | 19.2 |
| 3.25 | 15.2 | 16.3 |

### Example 8

### ζ-Potentials of Pea Protein Solution

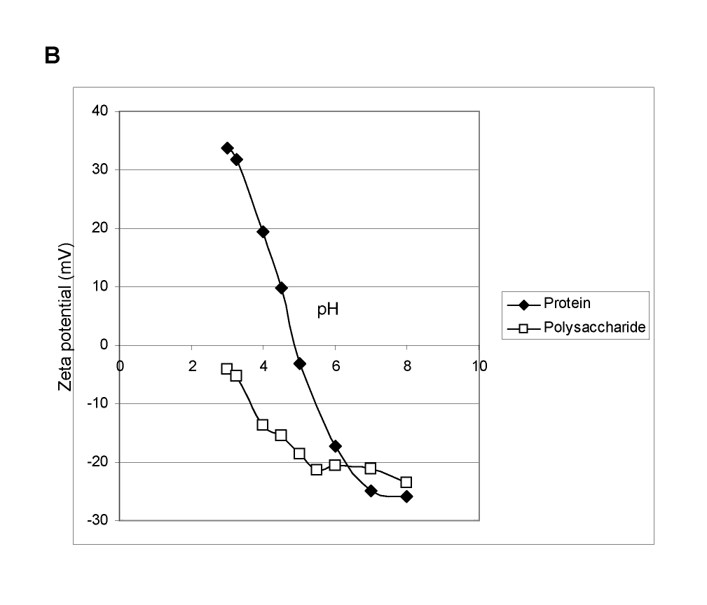

The ζ-potentials of pea protein solution before and after centrifugation at 5000 rpm are not significantly different (FIG. 2A). FIG. 2B shows the ζ-potentials of pea protein and soy polysaccharide solutions. The zero ζ-potential of pea protein is about pH 4.8. Pea protein and soy polysaccharide carries opposite charges in the pH range of 3.0 to 4.8, in this pH range, pea protein and soy polysaccharide can form electrostatic complexes.

### Example 9

### Pea Protein/Soy Polysaccharide Complex Emulsions Prepared From Pea Protein Solution Without Centrifugation

The polysaccharide stock solution of pH 3.25 was diluted with the same pH aqueous solution followed by 0.5 h stirring. Then, pH 3.25 pea protein stock solution (without centrifugation) was added. The final protein concentration in the mixed solution was 5 mg/mL, the weight ratio of protein to polysaccharide (WR) was 1:5. After the mixed aqueous solution was stirred for 3.5 h, soybean oil was added to reach a volume fraction of 10%. The mixture was pre-emulsified using a homogenizer (FJ200-S, Shanghai Specimen Model Co.) at 10000 rpm for 1 minute, and was immediately emulsified using a high pressure homogenizer (AH100D, ATS Engineering Inc.) at 850 bar for 4 min, followed by a heat treatment at 80° C. for 1 h. After overnight storage at 4° C., the resultant emulsions were adjusted to different pH values and NaCl was added. The emulsions containing designed pH value and NaCl concentration were stored at 4° C. to investigate the stability. The dynamic light scattering (DLS) result of the emulsions is shown in Table 9. After 26 days of storage, the emulsions were homogeneous. After further storage, the emulsions in the media containing 0.2 M NaCl presented creaming. The emulsions in pH 5 and 6 media without salt presented a little whey layer at the bottom. The whey layer is less than 10% compared to the whole emulsion volume after 180 days of storage.

TABLE 9
DLS result of the complex emulsions prepared at pH 3.25 from pea protein
without centrifugation at start of experiment and after 26 days storage.
The emulsion was heated at 80° C. for 1 h. The experiment was
repeated with two different batches labelled (1) and (2) to assess
reproducibility of the data.

| Sample | | Intensity | Dₕ (nm) | PDI |
|---|---|---|---|---|
| pH 3.25 | (1) | 40 | 284 | 0.14 |
| (emulsifying pH) | (2) | 36 | 280 | 0.21 |
| pH 5 | (1) | 37 | 319 | 0.25 |
| | (2) | 40 | 309 | 0.27 |
| pH 6 | (1) | 38 | 321 | 0.26 |
| | (2) | 43 | 306 | 0.23 |
| pH 5 + 0.2M NaCl | (1) | 36 | 312 | 0.27 |
| | (2) | 37 | 325 | 0.29 |
| pH 6 + 0.2M NaCl | (1) | 36 | 316 | 0.26 |
| | (2) | 32 | 322 | 0.27 |

After 26 days' storage:

| Storage condition | | Intensity | Dₕ (nm) | PDI |
|---|---|---|---|---|
| pH 3.25 | (1) | 55 | 275 | 0.13 |
| (emulsifying pH) | (2) | 52 | 264 | 0.10 |
| pH 5 | (1) | 57 | 320 | 0.19 |
| | (2) | 55 | 315 | 0.22 |
| pH 6 | (1) | 55 | 353 | 0.20 |
| | (2) | 41 | 341 | 0.16 |
| pH 5 + 0.2M NaCl | (1) | 33 | 379 | 0.22 |
| | (2) | 32 | 361 | 0.22 |
| pH 6 + 0.2M NaCl | (1) | 39 | 414 | 0.28 |
| | (2) | 39 | 380 | 0.20 |

We changed the heating temperature to 90° C. for 1 h in order to make the oil-water interfacial films more stable. The other emulsifying condition is the same as described above. After emulsifying and heating at pH 3.25, the emulsion was changed to pH 4, 5, 6, and NaCl was added to investigate the stability. After 94 days of storage in the pH range of 3.25 to 6, the emulsions were homogenous in appearance and the droplet sizes are smaller than 350 nm (Table 10). For the emulsions stored in the salt media for 94 days, creaming appeared. This result demonstrates that the emulsions are stable in the pH range of 3.25 to 6 that can be used in saltless beverages. In the following study the emulsions were heated at 90° C. for 1 h.

Besides emulsifying at pH 3.25, the emulsifying was also performed at pH 3.0, 3.5, 3.75, and 4.0. The resultant emulsions produced at the pH range of 3.5 to 4.0 are not stable because of the lower solubility of the pea protein in this pH range. The emulsion produced at pH 3.0 is not as stable as pH 3.25.

TABLE 10
DLS result of the complex emulsions prepared at pH 3.25 from pea protein
without centrifugation. The emulsion was heated at 90° C. for 1 h.
Results are shown for freshly prepared material and after 94 days.

| | Storage | | | |
|---|---|---|---|---|
| Sample | condition | Intensity | Dₕ (nm) | PDI |
| pH 3.25 | Freshly prepared | 58 | 265 | 0.15 |
| (emulsifying pH) | After 94 days | 29 | 257 | 0.15 |
| pH 4 | Freshly prepared | 59 | 260 | 0.18 |
| | After 94 days | 25 | 241 | 0.16 |
| pH 5 | Freshly prepared | 53 | 273 | 0.16 |
| | After 94 days | 28 | 289 | 0.09 |
| pH 6 | Freshly prepared | 58 | 309 | 0.18 |
| | After 94 days | 28 | 335 | 0.10 |
| pH 4 + 0.2M | Freshly prepared | 46 | 286 | 0.20 |
| NaCl | After 94 days | Creaming | | |
| pH 5 + 0.2M | Freshly prepared | 46 | 313 | 0.20 |
| NaCl | After 94 days | Creaming | | |
| pH 6 + 0.2M | Freshly prepared | 41 | 320 | 0.21 |

| | | |
|---|---|---|
| NaCl | After 94 days | Creaming |

### Example 10

### Pea Protein/Soy Polysaccharide Complex Emulsions Prepared From Pea Protein Solution Centrifuged At pH 3.25

In order to produce stable emulsion in salt medium, we then produced emulsions from protein solution that had been centrifuged at pH 3.25 to remove indiscerptible aggregates. The final protein concentration in aqueous solution is approximately 4 mg/mL, and the polysaccharide was 25 mg/mL. The other condition is the same as above. The droplet sizes of the resultant emulsions are shown in Table 11. The emulsions were homogenous after 87 days of storage in pH 5 and 6 with and without 0.2 M NaCl; the droplet size (Dₕ) does not change significantly after the storage in different media.

TABLE 11
DLS results of the complex emulsions prepared at pH 3.25 from two given
samples. Samples were measured freshly prepared and after 87 days storage.

| | | Sample 1^{a} | | | Sample 2^{b} | | |
|---|---|---|---|---|---|---|---|
| | | | Dₕ | | | Dₕ | |
| Sample | Storage | Intensity | (nm) | PDI | Intensity | (nm) | PDI |
| pH 3.25 | Fresh | 58 ± 7 | 260 ± 3 | 0.12 ± 0.02 | 47 ± 7 | 269 ± 0 | 0.17 ± 0.02 |
| (emulsifying | 87 days | 35 ± 3 | 287 ± 1 | 0.18 ± 0.02 | 33 ± 2 | 294 ± 1 | 0.19 ± 0.01 |
| pH) | | | | | | | |
| pH 5 | Fresh | 46 ± 12 | 258 ± 1 | 0.16 ± 0.06 | 54 ± 8 | 266 ± 5 | 0.17 ± 0.01 |
| | 87 days | 33 ± 2 | 289 ± 9 | 0.13 ± 0.07 | 31 ± 2 | 303 ± 6 | 0.16 ± 0.01 |
| pH 6 | Fresh | 46 ± 13 | 296 ± 3 | 0.12 ± 0.01 | 52 ± 6 | 300 ± 3 | 0.14 ± 0.01 |
| | 87 days | 29 ± 1 | 290 ± 2 | 0.22 ± 0.02 | 31 ± 3 | 324 ± 9 | 0.18 ± 0.01 |
| pH 5 + 0.2M | Fresh | 38 ± 2 | 276 ± 0 | 0.16 ± 0.03 | 34 ± 0 | 276 ± 4 | 0.16 ± 0.02 |
| NaCl | 87 days | 22 ± 0 | 262 ± 11 | 0.28 ± 0.02 | 27 ± 2 | 277 ± 5 | 0.17 ± 0.02 |
| pH 6 + 0.2M | Fresh | 42 ± 5 | 278 ± 5 | 0.17 ± 0.04 | 38 ± 3 | 280 ± 5 | 0.17 ± 0.02 |
| NaCl | 87 days | 21 ± 1 | 271 ± 16 | 0.25 ± 0.05 | 28 ± 0 | 311 ± 3 | 0.21 ± 0.01 |

^{a}The protein stock solution with a concentration of 50 mg/mL was adjusted to pH 3.25 then centrifugation at 5000 rpm for 30 min.
^{b}The centrifugation was carried out at 7800 rpm instead of 5000 rpm.

### Example 11

### Pea Protein/Soy Polysaccharide Complex Emulsions Prepared From Pea Protein Solution Centrifuged At pH 6.8.

In order to increase the utilization rate of the pea protein and also obtain stable emulsion in salt medium, we changed centrifugation pH. The original pea protein solution is pH 6.8, and original soy polysaccharide solution is pH 5.3. We centrifuged the protein solution at pH 6.8, at this pH the protein utilization rate is about 73%. Then we mixed the protein solution with soy polysaccharide solution which was pH 5.3 (without pH adjusting), or mixed them at pH 7.0 (with pH adjusting). The protein/polysaccharide mixture was further adjusted to pH 3.75. Table 12 shows no precipitates at pH 3.75, indicating the polysaccharide can protect the protein from precipitation. The mixture mixed at pH 7.0 then changed to pH 3.75 has smaller particle size and larger intensity, indicating a better complexation between the protein and polysaccharide. When mixing at pH 5.3, the protein aggregates can inhibit the protein binding with the polysaccharide. The mixtures in Table 12 were used to produce emulsions at pH 3.25; the droplet size is shown in Table 13. As the pea protein/soy polysaccharide complex solution mixed at pH 7.0 produced smaller droplets, we adopted the following condition to produce emulsions in the following study: pea protein solution of pH 6.8 was centrifuged at 5000 rpm for 30 min, the pH of the resultant supernatant and the pH of soy polysaccharide solution were adjusted to pH 7.0, respectively, followed by mixing the protein and polysaccharide solutions at pH 7.0, then changing the mixture to emulsifying pH.

TABLE 12
DLS results of complex solution at pH 3.75. The protein solution was
centrifuged at pH 6.8. The protein and polysaccharide solutions were mixed at different
pH.

| | Protein | Polysaccharide | | | | |
|---|---|---|---|---|---|---|
| | concentration | concentration | | | | |
| Sample | (mg/mL)^{a} | (mg/mL)^{a} | WR^{b} | Intensity | Dₕ (nm) | PDI |
| Mixture - | 5 | 25 | 1:5 | 152 ± 12 | 759 ± 57 | 1.00 |
| adjusted^{c} | | | | | | |
| Mixture - | | | | 139 ± 16 | 1460 ± 51 | 1.00 |
| unadjusted^{d} | | | | | | |

^{a}The concentration of DLS samples.
^{b}The weight ratio of protein to polysaccharide.
^{c}Both the protein and polysaccharide solutions were adjusted to pH 7.0 before mixing.
^{d}The pH was unadjusted before mixing.

TABLE 13
DLS results of the emulsions prepared at pH 3.25 from the
mixtures shown in Table 12.

| Emulsified pH | Sample | Intensity | Dₕ (nm) | PDI |
|---|---|---|---|---|
| 3.25 | Adjusted | 29 ± 3 | 301 ± 9 | 0.20 ± 0.02 |
| | Unadjusted | 28 ± 2 | 319 ± 10 | 0.22 ± 0.05 |

We further investigated the complex solution with different weight ratios of pea protein to soy polysaccharide (WR). The data in Table 14 further support that the complexation can destroy the aggregates of individual protein and individual polysaccharide, forming smaller complex particles.

TABLE 14
DLS results of protein, polysaccharide and protein/polysaccharide
complex solutions at pH 3.75. Both the protein and polysaccharide
solutions were adjusted to pH 7.0 before mixing; then the mixture
was changed to pH 3.75.

| | | Poly- | | | |
|---|---|---|---|---|---|
| | Protein | saccharide | | | |
| | concen- | con- | | | |
| | tration | centration | | | |
| Sample | (mg/mL) | (mg/mL) | Intensity | Dₕ (nm) | PDI |
| protein | 5 | 0 | 126 ± 3 | 1122 ± 48 | 0.52 ± 0.48 |
| poly- | 0 | 25 | 30 ± 5 | 704 ± 2 | 1.00 |
| sac- | | | | | |
| charide | | | | | |
| WR 2:1 | 5 | 2.5 | 145 ± 12 | 178 ± 2 | 0.60 ± 0.01 |
| WR 1:1 | 5 | 5 | 146 ± 16 | 190 ± 7 | 0.59 ± 0.08 |
| WR 1:2 | 5 | 10 | 136 ± 21 | 334 ± 59 | 0.81 ± 0.08 |
| WR 1:3 | 5 | 15 | 130 ± 9 | 397 ± 42 | 0.62 ± 0.10 |
| WR 1:4 | 5 | 20 | 127 ± 8 | 590 ± 129 | 0.92 ± 0.04 |
| WR 1:5 | 5 | 25 | 121 ± 9 | 741 ± 137 | 0.82 ± 0.18 |
| WR 1:6 | 5 | 30 | 134 ± 17 | 928 ± 110 | 0.92 ± 0.08 |

### Example 12

### Pea Protein/Soy Polysaccharide Complex Emulsions Prepared From Pea Protein Solution Centrifuged At pH 6.8. The Pea Protein And Soy Polysaccharide Solutions Were Adjusted To pH 7.0 Before Mixing

In the following study, the producing procedure of the emulsions is as follows. Pea protein aqueous solution (pH 6.8) was centrifuged at 5000 rpm for 30 min. Pea protein and soy polysaccharide solutions were adjusted to pH 7.0, respectively, then were mixed and stirred for 2 h. The mixture was further adjusted to emulsifying pH. After stirring for another 4 h, soybean oil was added to 10% volume fraction. The mixture was pre-emulsified using a homogenizer at 10000 rpm for 1 minute, and was immediately emulsified using a high pressure homogenizer at 800 bar for 3 min, followed by a heat treatment at 90° C. for 1 h. After overnight storage at 4° C., the resultant emulsions were adjusted to different pH values and NaCl was added. The emulsions containing designed pH value and NaCl concentration were stored at 4° C. to investigate the stability.

### (1) Influence of Emulsifying pH

The complex emulsions were produced in the pH range of 2.5 to 7. DLS results (Table 15) indicate that the emulsions produced at the pH range of 3.5 to 4.25 are stable at pH 5 and 6 media with 0.2 M NaCl; the emulsions were homogenous after the storage. At the pH range of 3.5 to 4.25, the electrostatic attraction is stronger between the protein and polysaccharide as shown in FIG. 2 that benefits the complexation. In the following study, we used pH 3.75 as the emulsifying pH.

TABLE 15
Droplet sizes of the complex emulsions produced at different pHs. The protein
concentration was 5 mg/mL and WR was 1:5 in aqueous solution. Measurements were
performed on freshly prepared material, and depending on the batches after 29, 37, or 52
days (d) storage.

| | | | | | pH 5 + | |
|---|---|---|---|---|---|---|
| Emulsifying | | As | | | 0.2M | pH 6 + |
| pH | Storage | prepared | pH 5 | pH 6 | NaCl | 0.2M NaCl |
| 2.50 | fresh | 732 ± 31 | 2372 ± 192 | 2042 ± 35 | 4686 ± 373 | 3995 ± 353 |
| 3.00 | fresh | 328 ± 6 | 331 ± 10 | 347 ± 6 | 818 ± 109 | 662 ± 2 |
| | 37 d | 305 | 336 | 293 | 337 | 536 |
| | 52 d | 301 | 288 | 375 | 534 | 463 |
| 3.25 | fresh | 299 ± 13 | 298 ± 13 | 324 ± 10 | 465 ± 54 | 442 ± 49 |
| | 37 d | 265 | 279 | 306 | 337 | 395 |
| | 52 d | 289 | 312 | 363 | 369 | 452 |
| 3.50 | fresh | 302 ± 18 | 299 ± 4 | 321 ± 5 | 383 ± 3 | 375 ± 3 |
| | 37 d | 264 | 308 | 300 | 330 | 389 |
| | 52 d | 291 | 308 | 359 | 411 | 429 |
| 3.75 | fresh | 292 ± 17 | 295 ± 11 | 314 ± 5 | 333 ± 9 | 346 ± 19 |
| | 37 d | 253 | 262 | 266 | 268 | 292 |
| | 52 d | 286 | 310 | 367 | 370 | 399 |
| 4.00 | fresh | 291 ± 15 | 309 ± 7 | 328 ± 15 | 360 ± 25 | 348 ± 9 |
| | 37 d | 245 | 270 | 284 | 270 | 318 |
| | 52 d | 300 | 327 | 382 | 403 | 437 |
| 4.25 | fresh | 288 ± 3 | 300 ± 1 | 321 ± 5 | 350 ± 1 | 352 ± 3 |
| | 37 d (1) | 268 | 281 | 313 | 345 | 422 |
| | 37 d (2) | 266 | 288 | 337 | 347 | 387 |
| 4.50 | fresh | 306 ± 8 | 326 ± 9 | 354 ± 8 | 473 ± 20 | 448 ± 18 |
| | 37 d (1) | 316 | 320 | 356 | 534 | 613 |
| | 37 d (2) | 303 | 291 | 322 | 501 | 532 |
| 5.00 | fresh | 555 ± 13 | 566 ± 6 | 597 ± 13 | 1156 ± 15 | 950 ± 0 |

| | | | | |
|---|---|---|---|---|
| 29 d (1) | 595 | 523 | 558 | Creaming |
| 29 d (2) | 621 | 600 | 501 | Creaming |

| | | | |
|---|---|---|---|
| 6.00 | fresh | Creaming | — |
| 7.00 | fresh | | |

### (2) Influence of the Weight Ratio of Pea Protein To Soy Polysaccharide (WR)

Fixing the protein concentration at 5 mg/mL and changing the polysaccharide concentration from 2.5 to 30 mg/mL, i.e., changing WR from 2:1 to 1:6. Table 16 indicates that when WR in the range of 1:2 to 1:6, the droplet size is not influenced by the environment significantly, suggesting the oil droplets have been covered by enough polysaccharide. We chose WR 1:4 and 1:5 for further study.

TABLE 16
DLS result of the complex emulsions produced at pH 3.75 with different WR.
The protein concentration was 5 mg/mL.

| | As prepared (pH 3.75) | | | Adjusted to pH 5 | | |
|---|---|---|---|---|---|---|
| Sample | Intensity | Dₕ (nm) | PDI | Intensity | Dₕ (nm) | PDI |
| WR2:1 | 16 ± 2 | 567 ± 9 | 0.54 ± 0.01 | 22 ± 2 | 624 ± 49 | 0.38 ± 0.23 |
| WR1:1 | 34 ± 8 | 367 ± 27 | 0.23 ± 0.01 | 38 ± 2 | 350 ± 10 | 0.15 ± 0.10 |
| WR1:2 | 53 ± 4 | 292 ± 11 | 0.12 ± 0.01 | 48 ± 1 | 279 ± 3 | 0.14 ± 0.01 |
| WR1:3 | 48 ± 8 | 284 ± 12 | 0.13 ± 0.03 | 48 ± 2 | 282 ± 5 | 0.14 ± 0.02 |
| WR1:4 | 48 ± 4 | 280 ± 13 | 0.12 ± 0.01 | 50 ± 4 | 284 ± 2 | 0.15 ± 0.07 |
| WR1:5 | 52 ± 8 | 288 ± 12 | 0.15 ± 0.01 | 52 ± 4 | 296 ± 4 | 0.15 ± 0.03 |
| WR1:6 | 57 ± 3 | 281 ± 11 | 0.14 ± 0.05 | 52 ± 3 | 298 ± 6 | 0.14 ± 0.03 |
| | Adjusted to pH 6 | | | Adjusted to pH 5 + 0.2M NaCl | | |
| Sample | Intensity | Dₕ (nm) | PDI | Intensity | Dₕ (nm) | PDI |
| WR2:1 | 19 ± 1 | 618 ± 24 | 0.64 ± 0.02 | 19 ± 3 | 2816 ± 344 | 1.00 ± 0 |
| WR1:1 | 36 ± 1 | 342 ± 14 | 0.20 ± 0.08 | 26 ± 2 | 1522 ± 689 | 0.62 ± 0.38 |
| WR1:2 | 49 ± 1 | 284 ± 1 | 0.09 ± 0.07 | 40 ± 2 | 358 ± 15 | 0.13 ± 0.08 |
| WR1:3 | 51 ± 3 | 301 ± 9 | 0.12 ± 0.05 | 44 ± 4 | 331 ± 9 | 0.10 ± 0.03 |
| WR1:4 | 49 ± 1 | 312 ± 14 | 0.12 ± 0.06 | 43 ± 2 | 326 ± 14 | 0.12 ± 0.07 |
| WR1:5 | 46 ± 1 | 323 ± 6 | 0.09 ± 0.07 | 44 ± 2 | 322 ± 8 | 0.10 ± 0.08 |
| WR1:6 | 54 ± 5 | 329 ± 14 | 0.12 ± 0.04 | 45 ± 2 | 310 ± 10 | 0.14 ± 0.07 |

| | Adjusted to pH 6 + 0.2M NaCl | | |
|---|---|---|---|
| Sample | Intensity | Dₕ (nm) | PDI |
| WR2:1 | 14 ± 1 | 1538 ± 369 | 0.70 ± 0.30 |
| WR1:1 | 26 ± 1 | 850 ± 251 | 58 ± 0.42 |
| WR1:2 | 42 ± 2 | 354 ± 9 | 0.13 ± 0.08 |
| WR1:3 | 40 ± 2 | 338 ± 13 | 0.12 ± 0.02 |
| WR1:4 | 43 ± 0 | 335 ± 18 | 0.14 ± 0.05 |
| WR1:5 | 38 ± 2 | 331 ± 11 | 0.15 ± 0.08 |
| WR1:6 | 45 ± 3 | 331 ± 15 | 0.12 ± 0.05 |

### (3) Influence of High Pressure Homogenization (HPH)

We changed homogenization pressure from 800 to 1200 bar. The data in Table 17 demonstrate the pressure does not have significant influence on the droplet size and stability of the emulsions. In the following study, we fixed HPH condition at 800 bar for 3 min.

TABLE 17
DLS result of the complex emulsions produced at pH 3.75 with different HPH
condition. The protein concentration was 5 mg/mL.

| | | pH 3.75 (As prepared) | | | Adjusted to pH 5 | | |
|---|---|---|---|---|---|---|---|
| Sample^{a} | WR | Intensity | Dₕ (nm) | PDI | Intensity | Dₕ (nm) | PDI |
| 800-3 | 1:4 | 47 ± 3 | 265 ± 1 | 0.14 ± 0.03 | 38 ± 8 | 278 ± 13 | 0.17 ± 0.02 |
| | 1:5 | 45 ± 2 | 281 ± 3 | 0.17 ± 0.03 | 30 ± 8 | 295 ± 3 | 0.16 ± 0.03 |
| 1000-3 | 1:4 | 52 ± 11 | 270 ± 6 | 0.16 ± 0.01 | 40 ± 4 | 280 ± 0 | 0.16 ± 0.02 |
| | 1:5 | 51 ± 1 | 275 ± 4 | 0.12 ± 0.04 | 34 ± 2 | 287 ± 1 | 0.15 ± 0.05 |
| 1200-3 | 1:4 | 44 ± 12 | 258 ± 1 | 0.12 ± 0.04 | 48 ± 8 | 264 ± 4 | 0.16 ± 0.04 |
| | 1:5 | 56 ± 2 | 266 ± 3 | 0.13 ± 0.03 | 38 ± 10 | 275 ± 0 | 0.16 ± 0.02 |
| | | Adjusted to pH 6 | | | Adjusted to pH 5 + 0.2M NaCl | | |
| Sample^{a} | WR | Intensity | Dₕ (nm) | PDI | Intensity | Dₕ (nm) | PDI |
| 800-3 | 1:4 | 42 ± 8 | 283 ± 5 | 0.14 ± 0.05 | 40 ± 6 | 311 ± 14 | 0.20 ± 0.02 |
| | 1:5 | 28 ± 9 | 311 ± 3 | 0.21 ± 0.07 | 37 ± 5 | 315 ± 3 | 0.19 ± 0.02 |
| 1000-3 | 1:4 | 36 ± 5 | 299 ± 2 | 0.18 ± 0.05 | 38 ± 2 | 306 ± 1 | 0.16 ± 0.02 |
| | 1:5 | 40 ± 7 | 306 ± 13 | 0.19 ± 0.02 | 40 ± 2 | 308 ± 2 | 0.12 ± 0.01 |
| 1200-3 | 1:4 | 48 ± 8 | 272 ± 5 | 0.12 ± 0.03 | 37 ± 5 | 302 ± 7 | 0.18 ± 0.01 |
| | 1:5 | 38 ± 14 | 282 ± 18 | 0.16 ± 0.02 | 40 ± 10 | 292 ± 2 | 0.16 ± 0.02 |

| | | Adjusted to pH 6 + 0.2M NaCl | | |
|---|---|---|---|---|
| Sample^{a} | WR | Intensity | Dₕ (nm) | PDI |
| 800-3 | 1:4 | 44 ± 6 | 314 ± 13 | 0.18 ± 0.02 |
| | 1:5 | 38 ± 8 | 332 ± 10 | 0.22 ± 0.03 |
| 1000-3 | 1:4 | 40 ± 4 | 312 ± 2 | 0.19 ± 0.04 |
| | 1:5 | 34 ± 2 | 321 ± 2 | 0.18 ± 0.01 |
| 1200-3 | 1:4 | 42 ± 4 | 298 ± 10 | 0.14 ± 0.02 |
| | 1:5 | 42 ± 1 | 305 ± 0 | 0.18 ± 0.03 |

^{a}Samples prepared by different HPH conditions. The figures represent the value of pressure intensity and the duration of HPH process. For instance, 800-3 indicates that the sample was homogenized at 800 bar for 3 min.

### (4) Influence of Heat Treatment

The emulsion produced at pH 3.75, 800 bar for 3 min from WR 1:4 complexes with a protein concentration of 5 mg/mL was divided into 2 parts. One was heated at 90° C. for 1 h, the other was not. Then, the emulsions were changed to pH 2 to 8 and 0.2 M NaCl was added. The data in Table 18 indicate that the heated emulsions are stable against pH and salt concentration changes. Heating can induce protein denaturation and form irreversible oil-water interfacial films composed of pea protein and soy polysaccharide. During the storage in different media, the unheated emulsions presented creaming in all the media containing salt, and also creaming in pH 7 and 8 media without salt. On the contrary, the heated emulsions are homogenous in all the media with and without salt. Table 18 also shows that the unheated emulsions are stable in the pH range of 3 to 5, the emulsions are homogenous, and the droplet sizes do not change after the storage. This result suggests that the unheated emulsion can encapsulate heat-sensitive lipophilic bioactive compounds and the emulsion can be used in saltless beverages.

TABLE 18
DLS results of the complex emulsions prepared at pH 3.75, 800 bar for 3 min
from WR 1:4 complexes with a protein concentration of 5 mg/mL. The heating was
performed at 90° C. for 1 h. Measurements were performed on freshly prepared material
and after 92 days storage.

| | | Unheated emulsions | | | Heated emulsions | | |
|---|---|---|---|---|---|---|---|
| Adjusted | | | Dₕ | | | Dₕ | |
| pH | Storage | Intensity | (nm) | PDI | Intensity | (nm) | PDI |
| pH 2 | fresh | 30 ± 3 | 390 ± 12 | 0.30 ± 0.04 | 30 ± 4 | 312 ± 12 | 0.22 ± 0.02 |
| | 92 days | Creaming | | | — | — | — |
| | 99 days | — | — | — | 41 ± 1 | 362 ± 3 | 0.26 ± 0.01 |
| pH 3 | fresh | 38 ± 2 | 271 ± 4 | 0.11 ± 0.03 | 37 ± 5 | 282 ± 6 | 0.17 ± 0.02 |
| | 92 days | 54 ± 1 | 289 ± 2 | 0.11 ± 0 | — | — | — |
| | 99 days | — | — | — | 49 ± 1 | 280 ± 4 | 0.15 ± 0.01 |
| pH 4 | fresh | 38 ± 4 | 270 ± 8 | 0.17 ± 0 | 38 ± 3 | 274 ± 4 | 0.18 ± 0.06 |
| | 92 days | 50 ± 5 | 304 ± 6 | 0.17 ± 0.02 | — | — | — |
| | 99 days | — | — | — | 49 ± 1 | 279 ± 1 | 0.17 ± 0.01 |
| pH 5 | fresh | 38 ± 4 | 280 ± 3 | 0.16 ± 0.01 | 36 ± 1 | 280 ± 8 | 0.23 ± 0.01 |
| | 92 days | 44 ± 1 | 327 ± 14 | 0.13 ± 0.01 | — | — | — |
| | 99 days | — | — | — | 50 ± 1 | 301 ± 2 | 0.20 ± 0 |
| pH 6 | fresh | 28 ± 3 | 364 ± 8 | 0.27 ± 0.10 | 37 ± 1 | 320 ± 25 | 0.26 ± 0.08 |
| | 92 days | Creaming | | | — | — | — |
| | 99 days | — | — | — | 45 ± 1 | 323 ± 1 | 0.15 ± 0.07 |
| pH 7 | fresh | 24 ± 1 | 495 ± 41 | 0.26 ± 0.03 | 34 ± 2 | 316 ± 14 | 0.17 ± 0.01 |
| | 92 days | Creaming | | | — | — | — |
| | 99 days | — | — | — | 50 ± 3 | 351 ± 2 | 0.21 ± 0.02 |
| pH 8 | fresh | 22 ± 6 | 514 ± 26 | 0.47 ± 0.07 | 38 ± 1 | 326 ± 10 | 0.17 ± 0.02 |
| | 92 days | Creaming | | | — | — | — |
| | 99 days | — | — | — | 37 ± 3 | 573 ± 8 | 0.24 ± 0.05 |
| pH 2 + 0.2M | fresh | 19 ± 0 | 1036 ± 152 | 1.00 ± 0 | 24 ± 2 | 342 ± 3 | 0.24 ± 0.02 |
| NaCl | 92 days | Creaming | | | — | — | — |
| | 99 days | — | — | — | 38 ± 1 | 400 ± 421 | 0.18 ± 0.02 |
| pH 3 + 0.2M | fresh | 26 ± 2 | 342 ± 12 | 0.20 ± 0.08 | 28 ± 1 | 312 ± 6 | 0.21 ± 0 |
| NaCl | 92 days | Creaming | | | — | — | — |
| | 99 days | — | — | — | 34 ± 3 | 344 ± 31 | 0.17 ± 0.05 |
| pH 4 + 0.2M | fresh | 30 ± 1 | 297 ± 1 | 0.20 ± 0.04 | 30 ± 0 | 293 ± 1 | 0.15 ± 0.01 |
| NaCl | 92 days | Creaming | | | — | — | — |
| | 99 days | — | — | — | 37 ± 1 | 338 ± 3 | 0.17 ± 0.01 |
| pH 5 + 0.2M | fresh | 21 ± 2 | 479 ± 28 | 0.41 ± 0.07 | 26 ± 0 | 316 ± 6 | 0.20 ± 0.05 |
| NaCl | 92 days | Creaming | | | — | — | — |
| | 99 days | — | — | — | 35 ± 2 | 355 ± 1 | 0.13 ± 0 |
| pH 6 + 0.2M | fresh | 20 ± 2 | 740 ± 70 | 0.74 ± 0.26 | 26 ± 2 | 320 ± 1 | 0.25 ± 0.04 |
| NaCl | 92 days | Creaming | | | — | — | — |
| | 99 days | — | — | — | 38 ± 2 | 364 ± 5 | 0.16 ± 0.04 |
| pH 7 + 0.2M | fresh | 20 ± 0 | 692 ± 24 | 0.62 ± 0.04 | 28 ± 2 | 323 ± 8 | 0.20 ± 0.07 |
| NaCl | 92 days | Creaming | | | — | — | — |
| | 99 days | — | — | — | 34 ± 0 | 343 ± 11 | 0.17 ± 0 |
| pH 8 + 0.2M | fresh | 15 ± 0 | 680 ± 44 | 0.96 ± 0.04 | 29 ± 0 | 336 ± 2 | 0.19 ± 0.01 |

| | | | | | |
|---|---|---|---|---|---|
| NaCl | 92 days | Creaming | — | — | — |

| | | | | |
|---|---|---|---|---|
| 99 days | — | — | — | Creaming |

### Example 13

### Pea Protein/Soy Polysaccharide Complex Emulsions Prepared From Pea Protein Solution Without Centrifugation. The Pea Protein And Soy Polysaccharide Solutions Were Adjusted To pH 7.0 Before Mixing

### 1) Emulsions Prepared From the Pea Protein Solution Without Centrifugation

Pea protein and soy polysaccharide solutions were adjusted to pH 7.0, respectively, then were mixed and stirred for 2 h. The protein concentration was 5 mg/mL and WR was 1:4. The mixture was further adjusted to emulsifying pH. After stirring for another 4 h, soybean oil was added to 10% volume fraction. The mixture was pre-emulsified using a homogenizer at 10000 rpm for 1 minute, and was immediately emulsified using a high pressure homogenizer at 800 bar for 4 min, followed by a heat treatment at 90° C. for 1 h. After overnight storage at 4° C., the resultant emulsions were adjusted to different pH values and NaCl was added. The emulsions containing designed pH value and NaCl concentration were stored at 4° C. to investigate the stability (Table 19). We further investigated the stability of the emulsions produced at pH 3.5 and 3.75 in different media (Table 20). The data in Table 19 and 20 indicate the emulsions prepared from uncentrifugated pea protein solution are also stable against pH and salt concentration changes.

TABLE 19
Droplet size of the complex emulsions prepared from the
pea protein solution without centrifugation.

| Emulsifying | As | | | pH 5 + | pH 6 + |
|---|---|---|---|---|---|
| pH | prepared | pH 5 | pH 6 | 0.2M NaCl | 0.2M NaCl |
| 3.0 | 261 | 300 | 309 | 381 | 349 |
| 3.25 | 265 | 301 | 306 | 349 | 345 |
| 3.5 | 263 | 294 | 305 | 320 | 305 |
| 3.75 | 236 | 319 | 327 | 328 | 323 |
| 4.0 | 245 | 316 | 320 | 345 | 329 |

TABLE 20
DLS results of the complex emulsions prepared at pH 3.5 from the pea protein
solution without centrifugation.
Heated emulsion:

| | Fresh heated emulsion | | | After 55 days of storage | | |
|---|---|---|---|---|---|---|
| Storage | | Dₕ | | | Dₕ | |
| condition | Intensity | (nm) | PDI | Intensity | (nm) | PDI |
| As | 32 ± 2 | 267 ± 6 | 0.16 ± 0.01 | 52 ± 2 | 278 ± 3 | 0.11 ± 0.01 |
| prepared | | | | | | |
| (pH 3.5) | | | | | | |
| pH 2 | 28 ± 2 | 326 ± 7 | 0.17 ± 0.03 | 41 ± 1 | 370 ± 6 | 0.22 ± 0.02 |
| pH 3 | 31 ± 1 | 276 ± 4 | 0.13 ± 0.01 | 49 ± 1 | 279 ± 1 | 0.16 ± 0.03 |
| pH 4 | 33 ± 2 | 250 ± 15 | 0.16 ± 0.03 | 48 ± 2 | 282 ± 1 | 0.14 ± 0.01 |
| pH 5 | 31 ± 1 | 300 ± 11 | 0.14 ± 0.04 | 46 ± 1 | 301 ± 3 | 0.14 ± 0.04 |
| pH 6 | 31 ± 1 | 306 ± 3 | 0.18 ± 0.02 | 48 ± 6 | 319 ± 4 | 0.20 ± 0.01 |
| pH 7 | 30 ± 0 | 327 ± 1 | 0.17 ± 0.02 | 41 ± 4 | 323 ± 16 | 0.19 ± 0.01 |
| pH 8 | 27 ± 5 | 311 ± 7 | 0.20 ± 0.01 | 45 ± 4 | 341 ± 6 | 0.21 ± 0.01 |
| pH 2 + 0.2M | 27 ± 0 | 322 ± 4 | 0.22 ± 0.01 | 29 ± 3 | 353 ± 13 | 0.17 ± 0.10 |
| NaCl | | | | | | |
| pH 3 + 0.2M | 29 ± 1 | 291 ± 0 | 0.16 ± 0.02 | 36 ± 1 | 321 ± 13 | 0.15 ± 0.01 |
| NaCl | | | | | | |
| pH 4 + 0.2M | 30 ± 3 | 280 ± 6 | 0.20 ± 0.03 | 38 ± 10 | 298 ± 13 | 0.11 ± 0.02 |
| NaCl | | | | | | |
| pH 5 + 0.2M | 27 ± 2 | 300 ± 2 | 0.16 ± 0.01 | 37 ± 11 | 336 ± 16 | 0.15 ± 0.04 |
| NaCl | | | | | | |
| pH 6 + 0.2M | 29 ± 1 | 300 ± 1 | 0.18 ± 0.01 | 34 ± 7 | 367 ± 12 | 0.21 ± 0 |
| NaCl | | | | | | |
| pH 7 + 0.2M | 29 ± 1 | 309 ± 8 | 0.20 ± 0.01 | 31 ± 3 | 438 ± 11 | 0.23 ± 0.04 |
| NaCl | | | | | | |
| pH 8 + 0.2M | 28 ± 0 | 307 ± 3 | 0.17 ± 0.02 | 36 ± 6 | 457 ± 18 | 0.26 ± 0.04 |
| NaCl | | | | | | |

Unheated emulsion:

| Storage | Fresh unheated emulsion | | | After 55 days of storage | | |
|---|---|---|---|---|---|---|
| condition | Intensity | Dₕ (nm) | PDI | Intensity | Dₕ (nm) | PDI |
| As | 32 ± 0 | 255 ± 2 | 0.15 ± 0.01 | 55 ± 1 | 287 ± 3 | 0.14 ± 0.01 |
| prepared | | | | | | |
| (pH 3.5) | | | | | | |
| pH 2 | 20 ± 2 | 529 ± 3 | 0.58 ± 0.05 | Creaming | | |
| pH 3 | 30 ± 2 | 263 ± 4 | 0.16 ± 0.04 | 55 ± 1 | 289 ± 1 | 0.13 ± 0.01 |
| pH 4 | 30 ± 5 | 250 ± 6 | 0.17 ± 0.02 | 54 ± 1 | 296 ± 4 | 0.17 ± 0.01 |
| pH 5 | 27 ± 3 | 303 ± 6 | 0.12 ± 0.05 | 50 ± 0 | 341 ± 4 | 0.21 ± 0.02 |

| | | | | |
|---|---|---|---|---|
| pH 6 | 21 ± 3 | 421 ± 24 | 0.22 ± 0.07 | Creaming |
| pH 7 | 17 ± 3 | 611 ± 147 | 0.59 ± 0.29 | |
| pH 8 | 15 ± 2 | 668 ± 229 | 0.75 ± 0.25 | |
| pH 2 + 0.2M | 14 ± 1 | 965 ± 110 | 1.0 ± 0 | |
| NaCl | | | | |
| pH 3 + 0.2M | 22 ± 4 | 411 ± 54 | 0.23 ± 0.05 | |
| NaCl | | | | |
| pH 4 + 0.2M | 26 ± 2 | 304 ± 6 | 0.23 ± 0.01 | |
| NaCl | | | | |
| pH 5 + 0.2M | 14 ± 3 | 604 ± 171 | 0.71 ± 0.29 | |
| NaCl | | | | |
| pH 6 + 0.2M | 14 ± 3 | 969 ± 400 | 0.79 ± 0.21 | |
| NaCl | | | | |

| | |
|---|---|
| pH 7 + 0.2M | Creaming |
| NaCl | |
| pH 8 + 0.2M | |
| NaCl | |

### 2) Emulsion Prepared With 20% And 30% Oil Volume Fraction

Pea protein solution without centrifugation and soy polysaccharide solution were adjusted to pH 7.0, respectively, then were mixed and stirred for 2 h. The protein concentration was 5 mg/mL and WR was 1:4. The mixture was further adjusted to pH 3.5, and then soybean oil was added to 20% and 30% volume fraction. The mixture was emulsified at 800 bar for 4 min, followed by a heat treatment at 90° C. for 1 h. The data in Table 21 indicate that the droplet size increases with the oil content, so, the stability of the emulsions decreases with the increase of oil content.

TABLE 21
DLS results of the emulsions prepared with 20% and 30% oil volume fraction
before and after 55 days of storage. The emulsions were heated at 90° C. for 1 h.
20% oil:

| | | freshly prepared | | | After 55 days storage | | |
|---|---|---|---|---|---|---|---|
| | | | Dₕ | | | Dₕ | |
| Sample | Batch | Intensity | (nm) | PDI | Intensity | (nm) | PDI |
| As prepared (pH 3.5) | (1) | 60 | 387 | 0.24 | 46 | 424 | 0.28 |
| ″ | (2) | 61 | 401 | 0.24 | 45 | 387 | 0.14 |
| pH 5 | (1) | 67 | 412 | 0.25 | 43 | 460 | 0.17 |
| ″ | (2) | 56 | 440 | 0.28 | 50 | 428 | 0.24 |
| pH 6 | (1) | 66 | 427 | 0.28 | 35 | 486 | 0.23 |
| ″ | (2) | 59 | 446 | 0.33 | 52 | 432 | 0.22 |
| pH 5 + 0.2M NaCl | (1) | 64 | 405 | 0.23 | 35 | 556 | 0.14 |
| ″ | (2) | 65 | 470 | 0.27 | 36 | 561 | 0.36 |
| pH 6 + 0.2M NaCl | (1) | 61 | 462 | 0.26 | 32 | 545 | 0.19 |
| ″ | (2) | 54 | 488 | 0.28 | 30 | 528 | 0.28 |

30% oil:

| | freshly prepared | | | After 3 days storage | | |
|---|---|---|---|---|---|---|
| Sample | Intensity | Dₕ (nm) | PDI | Intensity | Dₕ (nm) | PDI |

| | | | | |
|---|---|---|---|---|
| As prepared (pH 3.5) | 57 | 576 | 0.28 | Creaming |
| pH 5 | 53 | 621 | 0.29 | |
| pH 6 | 49 | 644 | 0.30 | |
| pH 5 + 0.2M NaCl | 50 | 678 | 0.37 | |
| pH 6 + 0.2M NaCl | 42 | 699 | 0.41 | |

### Example 14

### Emulsifying Ability And Stability of Individual Pea Protein

Individual pea protein solution with a concentration of 5 mg/mL was adjusted to pH 3, 4, 5, 6, and 7, followed by emulsification. The emulsions produced at pH 4, 5, and 6 presented creaming immediately after the emulsification. For the emulsion produced at pH 3 and 7, the droplet size is 290 and 358 nm before the heating, 273 and 398 nm after the heating, respectively. The heated and unheated emulsions produced at pH 3 and 7 were changed to pH 2 to 8 and NaCl was added. DLS result showed the droplet sizes increase acutely for fresh prepared samples (data not shown); creaming appeared for all the samples within one week except for the emulsion produced at pH 3.0 without pH change and salt addition.

The result above supports the conclusion that pea protein/soy polysaccharide complex emulsions are superior to individual pea protein and individual soy polysaccharide emulsions.

### Example 15

### Encapsulation of β-Carotene In the Droplets of Complex Emulsion

β-Carotene was added in soybean oil with a concentration of 1 mg/mL. The oil phase was heated at 70° C. for 2 h under nitrogen atmosphere to dissolve β-carotene. The emulsion was prepared at pH 3.75, 800 bar for 4 min with 10% oil volume fraction, 5 mg/mL pea protein and 20 mg/mL polysaccharide aqueous phase. After emulsification, the β-carotene was encapsulated into the droplets; the β-carotene was 0.1 mg/mL in the emulsion. The resultant emulsion was adjusted to different pH to investigate the stability. Although all the emulsions are homogenous in appearance after 11 days of storage in different pH, DLS result shown in Table 22 indicates that the encapsulated emulsions are stable in the pH range of 3, 4, and 5, where the droplet sizes do not change significantly.

TABLE 22
DLS result of the emulsion encapsulated β-carotene. The emulsion was
produced at pH 3.75, and β-carotene concentration was 0.1 mg/mL.

| Sample | Storage | Intensity | Dₕ (nm) | PDI |
|---|---|---|---|---|
| pH 3.75 | fresh | 28 ± 2 | 258 ± 1 | 0.15 ± 0.03 |
| (emulsifying pH) | 86 days | 58 ± 1 | 301 ± 4 | 0.12 ± 0.01 |
| pH 2 | fresh | 24 ± 0 | 441 ± 4 | 0.27 ± 0.02 |
| | 86 days | Creaming | | |
| pH 3 | fresh | 36 ± 1 | 272 ± 1 | 0.18 ± 0.01 |
| | 86 days | 56 ± 1 | 296 ± 2 | 0.14 ± 0.02 |
| pH 4 | fresh | 36 ± 1 | 265 ± 1 | 0.18 ± 0.01 |
| | 86 days | 61 ± 3 | 310 ± 5 | 0.16 ± 0.0 |
| pH 5 | fresh | 30 ± 0 | 285 ± 3 | 0.09 ± 0.03 |
| | 86 days | 56 ± 4 | 334 ± 4 | 0.23 ± 0.06 |
| pH 6 | fresh | 25 ± 1 | 365 ± 2 | 0.19 ± 0.01 |
| | 86 days | Creaming | | |
| pH 7 | fresh | 20 ± 1 | 614 ± 4 | 0.38 ± 0.01 |
| | 86 days | Creaming | | |
| pH 8 | fresh | 24 ± 1 | 551 ± 10 | 0.38 ± 0.01 |

| | |
|---|---|
| 86 days | Creaming |

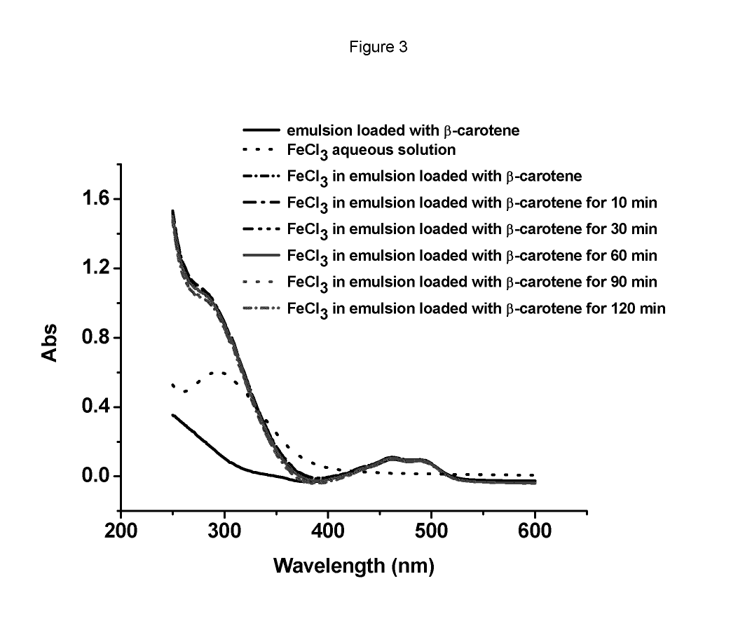

To further demonstrate the stability of the encapsulated β-carotene, the visible spectra of β-carotene emulsions were measured before and after the addition of FeCl₃ (see FIG. 3). For this experiment, we incubated the encapsulated material with FeCl₃. An emulsion was prepared by mixing 5 μl beta-carotene emulsion with 3 ml water, to which 20 μl of FeCl₃ at a concentration of 10 mg/ml were added. The final β-carotene concentration was 1.67×10⁻³ mg/mL. The blank solution for the measurement contains the same components with the same concentrations but without β-carotene. This experiment showed that the characteristic absorption band of β-carotene emulsion does not change after the addition of FeCl₃ demonstrating that the loaded β-carotene cannot react with FeCl₃, thereby confirming that the emulsion can protect β-carotene from oxidation during the storage.

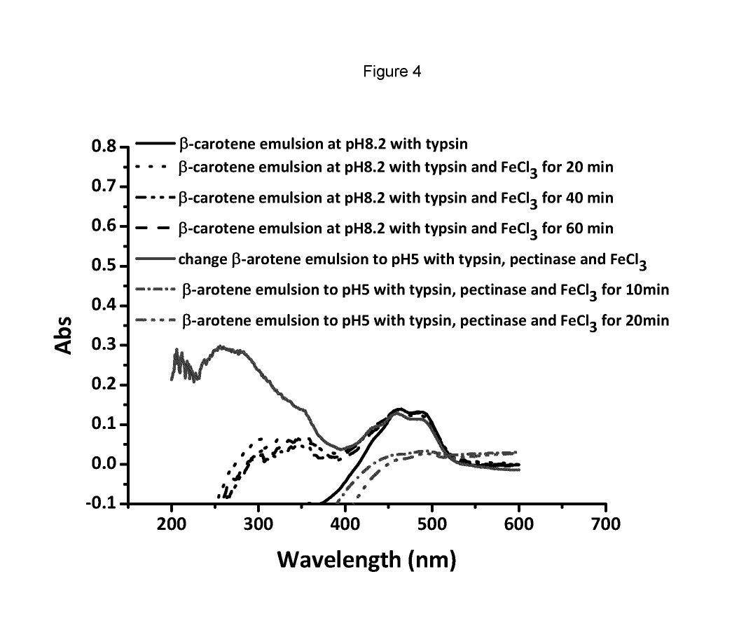

Furthermore, in order to measure the activity of the β-carotene emulsion, β-carotene was released by digestion of soy polysaccharide and pea protein using pectinase and typsin, respectively. The process is as follows: β-Carotene emulsion of 5 μL was added into 2.6 mL pH 8.2 Tris buffer (20 mM), and 400 μL typsin solution prepared by dissolving typsin in the Tris buffer with typsin concentration of 2 mg/mL was added. The β-carotene concentration in the mixture was 1.67×10⁻³ mg/mL. After visible spectrum measurement, 20 μL 10 mg/mL FeCl₃ was added into the mixture. These results (FIG. 4) show that the characteristic absorption band of β-carotene does not change in the presence of FeCl₃. Then, the mixture was adjusted to pH 5.0, and 40 μL pectinase solution and 20 μL 10 mg/mL FeCl₃ was added into the mixture. The visible spectra then showed that the characteristic absorption band of β-carotene disappears, demonstrating that the released β-carotene can react with FeCl₃. During the spectrum measurement, the blank solution contains the same components with the same concentrations but without β-carotene. Our control experiment confirmed that in β-carotene emulsion, the characteristic absorption band of β-carotene does not change in the presence of FeCl₃ when adding typsin or pectinase only.

### Example 16

### Encapsulation of Vitamin E In the Droplets of Complex Emulsion

Firstly we used pure vitamin E as oil phase to produce emulsions. The emulsion was prepared at pH 3.25, 800 bar for 3 min with 10% oil volume fraction, 5 mg/mL pea protein and 20 mg/mL polysaccharide aqueous phase. The emulsion was heated at 90° C. for 1 h. The DLS result shown in Table 23 indicates the emulsions are not stable in salt media.

TABLE 23
DLS result of the emulsions prepared at pH 3.25. Vitamin E
with a volume fraction of 10% was used as oil phase.

| Sample | Storage | Intensity | Dₕ (nm) | PDI |
|---|---|---|---|---|
| pH 3.25 (emulsifying pH) | fresh | 67 | 317 | 0.16 |
| | 37 days | 26 | 297 | 0.18 |
| pH 5 | fresh | 55 | 296 | 0.13 |
| | 37 days | 24 | 307 | 0.17 |
| pH 6 | fresh | 44 | 357 | 0.10 |
| | 37 days | 23 | 350 | 0.31 |
| pH 5 + 0.2M NaCl | fresh | 33 | 1827 | 1.0 |
| | 37 days | Creaming | | |
| pH 6 + 2M NaCl | fresh | 29 | 1060 | 1.0 |

| | |
|---|---|
| 37 days | Creaming |

Considering the viscosity of vitamin E, the oil phase was changed to 40% vitamin E and 60% soybean oil mixture. The other condition is the same as above. The emulsion was produced at pH 3.75. The data in Table 24 show the droplet sizes are not sensitive to the pH and salt concentration changes, implying the emulsions will be stable in these media.

TABLE 24
DLS result of the emulsions prepared at pH 3.75 with an oil volume fraction of
10%. The oil phase was composed of 40% vitamin E and 60% soybean oil.

| | Fresh heated emulsion | | | After 55 days of storage | | |
|---|---|---|---|---|---|---|
| | | Dₕ | | | Dₕ | |
| Storage condition | Intensity | (nm) | PDI | Intensity | (nm) | PDI |
| As prepared (pH | 30 ± 0 | 284 ± 1 | 0.13 ± 0 | 65 ± 5 | 298 ± 10 | 0.14 ± 0.01 |
| 3.75) | | | | | | |
| pH 2 | 28 ± 3 | 350 ± 35 | 0.25 ± 0.02 | 38 ± 7 | 344 ± 3 | 0.22 ± 0.01 |
| pH 3 | 30 ± 1 | 284 ± 5 | 0.17 ± 0.02 | 64 ± 7 | 297 ± 5 | 0.14 ± 0.02 |
| pH 4 | 31 ± 2 | 279 ± 2 | 0.20 ± 0.03 | 64 ± 5 | 300 ± 5 | 0.16 ± 0.02 |
| pH 5 | 32 ± 1 | 316 ± 16 | 0.20 ± 0.01 | 57 ± 8 | 293 ± 9 | 0.16 ± 0.02 |
| pH 6 | 29 ± 0 | 330 ± 17 | 0.23 ± 0.06 | 60 ± 10 | 378 ± 31 | 0.28 ± 0.07 |
| pH 7 | 29 ± 1 | 342 ± 5 | 0.26 ± 0.01 | 62 ± 6 | 342 ± 13 | 0.21 ± 0.03 |
| pH 8 | 31 ± 1 | 344 ± 20 | 0.21 ± 0.02 | 59 ± 9 | 380 ± 2 | 0.23 ± 0.02 |
| pH 2 + 0.2M NaCl | 26 ± 2 | 364 ± 13 | 0.34 ± 0.06 | 50 ± 0 | 415 ± 12 | 0.23 ± 0.02 |
| pH 3 + 0.2M NaCl | 27 ± 2 | 329 ± 7 | 0.23 ± 0.02 | 51 ± 7 | 398 ± 41 | 0.25 ± 0.05 |
| pH 4 + 0.2M NaCl | 27 ± 2 | 292 ± 10 | 0.21 ± 0.03 | 60 ± 2 | 363 ± 13 | 0.20 ± 0.01 |
| pH 5 + 0.2M NaCl | 28 ± 2 | 323 ± 2 | 0.19 ± 0.02 | 58 ± 1 | 400 ± 33 | 0.24 ± 0.01 |
| pH 6 + 0.2M NaCl | 27 ± 3 | 324 ± 1 | 0.26 ± 0.02 | 53 ± 1 | 363 ± 5 | 0.20 ± 0.07 |

| | | | | |
|---|---|---|---|---|
| pH 7 + 0.2M NaCl | 32 ± 2 | 317 ± 2 | 0.25 ± 0.03 | Creaming |
| pH 8 + 0.2M NaCl | 32 ± 3 | 322 ± 3 | 0.23 ± 0.01 | |

### Example 17

### Influence of the Weight Ratio of Soy Protein To Soy Polysaccharide (WR) On the Stability of Soy Protein/Soy Polysaccharide Emulsions

Table 25 shows soy protein/soy polysaccharide emulsions prepared from different weight ratios of soy protein to polysaccharide (WR). The data in Table 24 reveal the emulsions prepared from WR 1:2 to 1:6 complexes are stable against pH and salt concentration changes as well as long-term storage.

TABLE 25
Influence of the weight ratio of soy protein to polysaccharide (WR) on the
droplet sizes of the emulsions in different media. The emulsions were produced at pH
3.25. The protein concentration was 5 mg/mL in aqueous solution, and oil volume fraction
was 10%.

| | | Dₕ (nm) | | | | |
|---|---|---|---|---|---|---|
| | | pH 3.25 | | | | |
| | | (emulsifying | | | pH 5 + 0.2M | pH 6 + 0.2M |
| Sample | | pH) | pH 5 | pH 6 | NaCl | NaCl |
| Freshly | WR 2:1 | 367 ± 15 | 379 ± 32 | 415 ± 38 | 2445 ± 345 | 2265 ± 489 |
| prepared | WR 1:1 | 302 ± 3 | 343 ± 28 | 348 ± 28 | 1184 ± 219 | 1168 ± 285 |
| | WR 1:2 | 265 ± 18 | 271 ± 6 | 292 ± 4 | 346 ± 81 | 391 ± 1 |
| | WR 1:3 | 255 ± 12 | 275 ± 8 | 289 ± 1 | 320 ± 50 | 326 ± 40 |
| | WR 1:4 | 263 ± 15 | 289 ± 6 | 302 ± 4 | 315 ± 21 | 325 ± 30 |
| | WR 1:5 | 273 ± 11 | 298 ± 1 | 312 ± 4 | 317 ± 11 | 322 ± 1 |
| | WR 1:6 | 272 ± 2 | 326 ± 12 | 342 ± 14 | 327 ± 11 | 342 ± 8 |
| | WR 1:7 | 297 ± 2 | 342 ± 2 | 381 ± 2 | 450 ± 1 | 498 ± 3 |
| After 45 | WR 2:1 | 390 ± 11 | Creaming | | | |
| days of | WR 1:1 | 305 ± 16 | | | | |
| storage | WR 1:2 | 277 ± 18 | 307 ± 6 | 294 ± 19 | 419 ± 165 | 409 ± 144 |
| | WR 1:3 | 265 ± 10 | 296 ± 6 | 303 ± 28 | 369 ± 109 | 336 ± 64 |
| | WR 1:4 | 274 ± 15 | 300 ± 5 | 307 ± 28 | 346 ± 61 | 351 ± 64 |
| | WR 1:5 | 275 ± 0 | 308 ± 6 | 320 ± 18 | 339 ± 37 | 353 ± 37 |
| | WR 1:6 | 280 ± 4 | 337 ± 12 | 345 ± 16 | 351 ± 26 | 365 ± 25 |

| | | |
|---|---|---|
| WR 1:7 | 304 ± 4 | Creaming |

### Example 18

### Influence of the Weight Ratio of Pea Protein To Soy Polysaccharide (WR) On the Stability of Pea Protein/Soy Polysaccharide Emulsions. Pea Protein Solution Without Centrifugation Was Used

Pea protein and soy polysaccharide solutions were adjusted to pH 7.0, respectively, then were mixed and stirred for 2 h. The protein concentration was 5 mg/mL and WR was changed from 1:1 to 1:6. The mixture was emulsifying at pH 3.5 with 10% oil fraction at 800 bar for 4 min and followed by a heat treatment at 90° C. for 1 h. The data in Table 26 confirm the suitable WR values are in the range of 1:2-1:6.

TABLE 26
Influence of weight ratio of pea protein to soy polysaccharide (WR)
on the droplet size of pea protein/soy polysaccharide complex
emulsions prepared from pea protein solution without centrifugation.

| | Storage condition | | | | |
|---|---|---|---|---|---|
| | As | | | pH 5 + 0.2M | pH 6 + 0.2M |
| WR | prepared | pH 5 | pH 6 | NaCl | NaCl |

Freshly prepared:

| | | | | | |
|---|---|---|---|---|---|
| 1:1 | 258 | 276 | 280 | 638 | 400 |
| 1:2 | 276 | 303 | 316 | 315 | 314 |
| 1:3 | 268 | 297 | 310 | 292 | 297 |
| 1:4 | 274 | 314 | 315 | 291 | 288 |
| 1:5 | 271 | 322 | 315 | 284 | 292 |
| 1:6 | 286 | 326 | 327 | 299 | 305 |

After 63 days of storage:

| 1:1 | 244 | 242 | 238 | Creaming | |
|---|---|---|---|---|---|
| 1:2 | 268 | 246 | 288 | 328 | 336 |
| 1:3 | 275 | 274 | 284 | 313 | 317 |
| 1:4 | 281 | 286 | 302 | 324 | 352 |
| 1:5 | 288 | 294 | 301 | 314 | 394 |
| 1:6 | 297 | 305 | 308 | 344 | 393 |

1. Composition comprising:
a) 0.1 to 70 weight-% based on the composition of one or more fat-soluble active ingredients;
b) one or more plant protein(s) chosen from the group of proteins suitable for food application; and
c) one or more soy soluble polysaccharide(s);
wherein the sum of the amount of protein(s) and the amount of polysaccharide(s) represents 10 to 85 weight-% based on the composition in dry matter and, wherein the weight ratio of protein(s) to polysaccharide(s) is chosen like 1:b with the proviso that b is comprised between 0.5 and 15.

2. Composition according to claim 1, characterized in that the fat-soluble active ingredient(s) (one or more compounds) are selected from the group consisting of vitamin A, D, E, K and derivatives thereof; polyunsaturated fatty acids; lipophilic health ingredients; carotenoids; and flavoring or aroma substances as well as mixtures thereof.

3. Composition according to claim 1, characterized in that fat-soluble active ingredient(s) (one or more compounds) are carotenoids, especially beta-carotene, lycopene, lutein, bixin, astaxanthin, apocarotenal, beta-apo-8′-carotenal, beta-apo-12′-carotenal, canthaxanthin, cryptoxanthin, citranaxanthin and/or zeaxanthin.

4. Composition according to claim 2, characterized in that the lipophilic health ingredient(s) (one or more compounds) are selected from the group consisting of resveratrol; ligusticum; ubichinones and/or ubiquinols (one or more components), preferred coenzyme Q 10, coenzyme Q 9, and/or their reduced forms (the corresponding ubiquinols); genistein and alpha-lipoic acid.

5. Composition according to claim 1, characterized in that the proteins are plant protein(s) (one or more compounds) selected from the group consisting of soy protein, lupin protein, pea protein and potato protein.

6. Composition according to claim 5, characterized in that the plant proteins is soy or pea protein.

7. Process for the manufacture of a stable emulsifier which comprises the following steps:
I) suspending the protein in water;
II) optionally removing not-dissolved protein from the suspension of step I);
III) mixing one or more soy soluble polysaccharide(s) in a weight ratio of protein(s) to polysaccharide(s) of from 1:0.5 to 1:15;
IV) adjusting the pH to a value comprised between 3 and 5
V) adding the organic phase, comprising the one or more fat-soluble active ingredients to the complex;
VI) homogenizing the mixture of step V) with a conventional emulsification process known to the person skilled in the art.
VII) heating the emulsion at a temperature comprised between 70 to 95° C., preferably 80 to 90° C. for at least 45 minutes, preferably, at least 1 hour VIII) optionally drying the emulsion of step VII).

8. Plant protein-soy soluble polysaccharide complex obtainable by a process according to claim 7.

9. Plant protein-soy soluble polysaccharide according to claim 8, wherein the plant protein is soy protein or pea protein.

10. Use of a composition as claimed in claim 1, for the enrichment, fortification and/or coloration of food, beverages, animal feed, cosmetics or pharmaceutical compositions.

11. Process for the manufacture of a beverage comprising the step of mixing a composition according to claim 1 with further usual ingredients of beverages.

12. Beverage obtainable by the process according to claim 11.
